(19) United States
(12) Patent Application Publication
(10) Pub. No.: US 2025/0134876 A1
(43) Pub. Date: May 1, 2025

(54) METHODS OF TREATING A CANCER OVEREXPRESSING ONE OR MORE BCL-2 FAMILY PROTEINS

(71) Applicant: Kartos Therapeutics, Inc., New York, NY (US)

(72) Inventor: Wayne Rothbaum, Delray Beach, FL (US)

(21) Appl. No.: 18/690,554

(22) PCT Filed: Sep. 9, 2022
(86) PCT No.: PCT/US2022/043054

§ 371 Date: Mar. 8, 2024

Related U.S. Application Data
(60) Provisional application No. 63/242,427, filed on Sep. 9, 2021.

(51) Int. Cl. A61K 31/451; A61K 45/06; A61P 35/00; A61P 35/02

(52) U.S. Cl. CPC A61K 31/451; A61K 45/06; A61P 35/00; A61P 35/02

(57) ABSTRACT

Therapeutic methods and pharmaceutical compositions for treating a cancer overexpressing one or more Bcl-2 anti-apoptotic/pro-survival family of proteins using a MDM2 inhibitor.

### FIELD OF THE INVENTION

Methods of treating a cancer overexpressing one or more Bcl-2 family proteins using a Mouse double minute 2 homolog (MDM2) inhibitor are disclosed herein.

### BACKGROUND OF THE INVENTION

p53 is a tumor suppressor and transcription factor that responds to cellular stress by activating the transcription of numerous genes involved in cell cycle arrest, apoptosis, senescence, and DNA repair. Unlike normal cells, which have infrequent cause for p53 activation, tumor cells are under constant cellular stress from various insults including hypoxia and pro-apoptotic oncogene activation. Thus, there is a strong selective advantage for inactivation of the p53 pathway in tumors, and it has been proposed that eliminating p53 function may be a prerequisite for tumor survival. In support of this notion, mouse models have been used to demonstrate that absence of p53 function is a continuous requirement for the maintenance of established tumors (Cheok, Seeking synergy in p53 transcriptional activation for cancer therapy. Discov Med., 2012, 14(77), 263-271; Qian, Tumor suppression by p53: making cells senescent. Histol Histopathol. 2010, 25(4), 515-26; Beraza, Restoration of p53 function: a new therapeutic strategy to induce tumor regression?Hepatology, 2007, 45(6), 1578-9). When the investigators restored p53 function to tumors with inactivated p53, the tumors regressed.

p53 is inactivated by mutation and/or loss in 50% of solid tumors and 10% of liquid tumors. Other key members of the p53 pathway are also genetically or epigenetically altered in cancer. For example, MDM2, an oncoprotein, inhibits p53 function, and amplification of MDM2 gene was found in more than a third of sarcomas and in a subset of malignant gliomas. In absence of gene amplification, the MDM2 gene was overexpressed in some types of leukemias and lymphomas (Watanabe, Overexpression of the MDM2 oncogene in leukemia and lymphoma. Leukemia and Lymphoma, 1996, 21, 391-7). MDM2, in turn, is inhibited by another tumor suppressor, p14ARF. It has been suggested that alterations downstream of p53 may be responsible for at least partially inactivating the p53 pathway in p53 WT tumors (p53 wild-type). In support of this concept, some p53WT tumors appear to exhibit reduced apoptotic capacity, although their capacity to undergo cell cycle arrest remains intact. One cancer treatment strategy involves the use of small molecules that bind MDM2 and neutralize its interaction with p53. MDM2 inhibits p53 activity by three mechanisms: (1) acting as an E3 ubiquitin ligase to promote p53 degradation; (2) binding to and blocking the p53 transcriptional activation domain; and (3) exporting p53 from the nucleus to the cytoplasm. All three of these mechanisms would be blocked by neutralizing the MDM2-p53 interaction. In particular, this therapeutic strategy could be applied to tumors that are p53 WT, and studies with small molecule MDM2 inhibitors have yielded promising reductions in tumor growth both in vitro and in vivo. Further, in patients with p53-inactivated tumors, stabilization of wild-type p53 in normal tissues by MDM2 inhibition might allow selective protection of normal tissues from mitotic poisons. As used herein, MDM2 means a human MDM2 protein and p53 means a human p53 protein. It is noted that human MDM2 can also be referred to as HDM2 or hMDM2. Several MDM2 inhibitors are in human clinical trials for the treatment of various cancers.

The Bcl-2 family of proteins plays a major role in tumorogenesis. Bcl-2 proteins are characterized based on the presence of Bcl-2 homology (BH) domains. The anti-apoptotic proteins contain all the BH1-4 domains; the pro-apoptotic proteins contain either the BH3 domain only or multiple BH domains. The anti-apoptotic Bcl-2 proteins interact with pro-apoptotic members and inhibit their function to maintain cellular homeostasis. It is the shift in balance between anti-apoptotic and pro-apoptotic Bcl-2 proteins that may decide the fate of cancer cells. Elevated BCL-2 gene expression is estimated to occur in perhaps as many as half of all human cancers (Yip, Bcl-2 family proteins and cancer. Oncogene, 2008, 27, 6398-6406).

In the mitochondria, p53 can bind to anti-apoptotic Bcl-2 family proteins Bcl-XL and Bcl-2 and, release the pro-apoptotic effectors (“pore-formers”) Bak/Bax that would otherwise be sequestered by the anti-apoptotic proteins. Thereby, the released Bak and Bax are free to oligomerize to induce lipid pore formation in the outer mitochondrial membrane, which elicits cytochrome c release and eventually leads to caspase-dependent cellular apoptosis (Tomita, WT p53, but not tumor-derived mutants, bind to Bcl2 via the DNA binding domain and induce mitochondrial permeabilization. J. Biol. Chem. 2006, 281, 8600-8606). Further, p53 can directly activate Bak and/or Bax through a “hit and run” mechanism to trigger the permeabilization of outer mitochondrial membrane leading to cell apoptosis (Chipuk, Direct activation of Bax by p53 mediates mitochondrial membrane permeabilization and apoptosis. Science, 2004, 303, 1010-1014). Within the nucleus, p53 induces transcription-dependent apoptosis via the transactivation of target genes such as Bcl-2 family pro-apoptotic genes: Noxa, Bax, and Puma. Transcription-dependent apoptosis induced by p53 is mediated via interactions of the p53 transactivation domain (p53TAD) with the components of the basal transcriptional machinery. Under normal conditions, the p53 level is controlled by mouse double minute 2 (MDM2), which is an E3 ubiquitin ligase that negatively regulates p53 stability through ubiquitination and inhibits the transactivation ability of p53 by interacting with p53TAD. Therefore, blocking the interaction of p53 with MDM2 is a promising anticancer approach (Chi, Structural insights into the transcription-independent apoptotic pathway of p53. BMB Rep. 2014, 47(3), 167-172).

The present invention provides methods of treating a cancer overexpressing one or more Bcl-2 family proteins in a human subject with a composition comprising an MDM2 inhibitor.

### SUMMARY OF THE INVENTION

The invention encompasses a method of treating a cancer overexpressing one or more Bcl-2 family proteins comprising the step of administering to a human subject in need thereof a therapeutically effective amount of a MDM2 inhibitor, wherein the MDM2 inhibitor is a compound of Formula (I) or Formula (II):

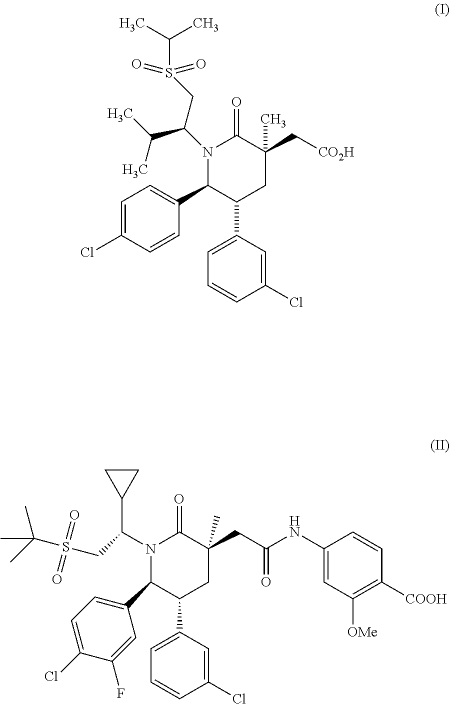 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1 ; COc1cc(NC(=O)C[C@@]2(C)C[C@H](c3cccc(Cl)c3)[C@@H](c3ccc(Cl)c(F)c3)N([C@H](CS(=O)(=O)C(C)(C)C)C3CC3)C2=O)ccc1C(=O)O]

or a pharmaceutically acceptable salt thereof.

In some embodiments, the MDM2 inhibitor is administered on days 1-7 of 21-day cycle, wherein on days 8-21 the MDM2 inhibitor is not administered. In some embodiments, the cancer overexpressing one or more Bcl-2 family proteins is selected from the group consisting of Merkel cell carcinoma, diffuse large B-cell lymphoma (DLBCL), mantle cell lymphoma (MCL), metastatic prostate cancer, small cell lung cancer (SCLC), Burkett's lymphoma, T-cell lymphoma, acute myeloid leukemia (AML) and myelofibrosis (MF). In an embodiment, the T-cell lymphoma is peripheral T-cell lymphoma or aggressive T-cell lymphoma. In an embodiment, the cancer is a relapsed/refractory cancer. In an embodiment, the small cell lung cancer is a p53 wild-type lung cancer. In some embodiments, the cancer overexpressing one or more Bcl-2 family proteins is a p53 wild-type cancer.

In some embodiments, the cancer overexpressing one or more Bcl-2 family proteins is a B cell hematological malignancy. In an embodiment, the B cell hematological malignancy is selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma and Waldenström's macroglobulinemia (WM).

In some embodiments, the cancer is relapsed/refractory (R/R). Relapsed means a cancer has re-occurred after no longer being detectable. Refractory means a cancer has stopped responding to treatment. In some embodiments, the cancer is relapsed/refractory chronic lymphocytic leukemia (R/R CLL), relapsed/refractory small lymphocytic leukemia (R/R SLL), relapsed/refractory non-Hodgkin's lymphoma (R/R NHL), relapsed/refractory diffuse large B cell lymphoma (R/R DLBCL), relapsed/refractory follicular lymphoma (R/R FL), relapsed/refractory mantle cell lymphoma (R/R MCL), relapsed/refractory Hodgkin's lymphoma, relapsed/refractory B cell acute lymphoblastic leukemia (R/R B-ALL), relapsed/refractory Burkitt's lymphoma, relapsed/refractory Waldenström's macroglobulinemia (R/R WM), relapsed/refractory acute myeloid leukemia (R/R AML) and relapsed/refractory myelofibrosis (R/R MF).

In some embodiments of the method, the compound of Formula (I) or Formula (II) is in a crystalline form. In some embodiments of the method, the compound of Formula (I) or Formula (II) is in a free form. In some embodiments of the method, the MDM2 inhibitor is a pharmaceutically acceptable salt of a compound of Formula (I) or Formula (II). In some embodiments of the method, the compound of Formula (I) or Formula (II) is in an amorphous form.

In some embodiments of the method, the compound of Formula (I) or Formula (II) is administered once daily at a dose selected from the group consisting of 15 mg, 25 mg, 30 mg, 50 mg, 60 mg, 75 mg, 90 mg, 100 mg, 120 mg, 150 mg, 175 mg, 180 mg, 200 mg, 225 mg, 240 mg, 250 mg, 275 mg, 300 mg, 325 mg, 350 mg, 360 mg, 375 mg, and 480 mg. In some embodiments of the method, the compound of Formula (I) or Formula (II) is administered twice daily at a dose selected from the group consisting of 15 mg, 25 mg, 30 mg, 50 mg, 60 mg, 75 mg, 90 mg, 100 mg, 120 mg, 150 mg, 175 mg, 180 mg, 200 mg, 225 mg, 240 mg, 250 mg, 275 mg, 300 mg, 325 mg, 350 mg, 360 mg, 375 mg, and 480 mg. In some embodiments of the method, the human subject is treated with the MDM2 inhibitor on days 1-7 of 21-day cycle, wherein on days 8-21 the human is not treated with the MDM2 inhibitor. In an embodiment, the compound of Formula (I) or Formula (II) is orally administered. In an embodiment, the therapeutically effective amount of the MDM2 inhibitor is 120 mg.

In an embodiment of the method, the human subject is previously treated with immunotherapy. In some embodiments, the immunotherapy is an ex vivo cell therapy selected from the group consisting of tumor-infiltrating lymphocytes (TILs), T-cell receptor (TCR)-engineered peripheral blood lymphocytes (PBL) and chimeric antigen receptor ((CAR)-engineered PBL). In some embodiments, the immunotherapy is an immune checkpoint protein inhibitor therapy. In some embodiments, the immune checkpoint protein inhibitor is an anti-PD-L1 antibody selected from the group consisting of BMS-936559, durvalumab, atezolizumab, avelumab, MPDL3280A, MEDI4736, MSB0010718C, MDX1105-01, and fragments, conjugates, biosimilars, or variants thereof. In some embodiments, the immune checkpoint protein inhibitor is an anti-PD-1 antibody selected from group consisting of nivolumab, tslelizimab, dostarlimab, pembrolizumab, pidilizumab, cemiplimab-rwlc, AMP-224, AMP-514, PDR001, and fragments, conjugates, biosimilars, or variants thereof. In some embodiments, the immune checkpoint protein inhibitor is an anti-CTLA-4 antibody selected from the group consisting of ipilimumab, tremelimumab, and fragments, conjugates, biosimilars, or variants thereof. In some embodiments, the immunotherapy is a T-cell engager selected from catumaxomab, FBTA05, Ertumaxomab, Ektomun, blinatumomab, solitomab, and fragments, conjugates, biosimilars, or variants thereof.

In an embodiment of the method, the human subject has Ki-67 protein proliferation rate more than 10%. In an embodiment of the method, the human subject has Ki-67 protein proliferation rate more than 15%. In an embodiment of the method, the human subject has Ki-67 protein proliferation rate more than 20%. In an embodiment of the method, the human subject has Ki-67 protein proliferation rate more than 25%.

The invention also relates to an MDM2 inhibitor for use in treating a cancer overexpressing one or more Bcl-2 family proteins, wherein the MDM2 inhibitor is a compound of Formula (I) or Formula (II):

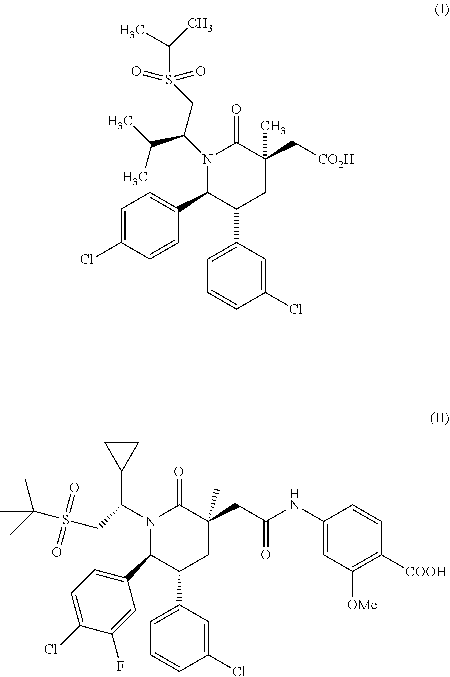 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1 ; COc1cc(NC(=O)C[C@@]2(C)C[C@H](c3cccc(Cl)c3)[C@@H](c3ccc(Cl)c(F)c3)N([C@H](CS(=O)(=O)C(C)(C)C)C3CC3)C2=O)ccc1C(=O)O]

or a pharmaceutically acceptable salt thereof.

In some embodiments, the cancer is selected from the group consisting of Merkel cell carcinoma, diffuse large B-cell lymphoma (DLBCL), mantle cell lymphoma (MCL), metastatic prostate cancer, small cell lung cancer (SCLC), Burkett's lymphoma, T-cell lymphoma, acute myeloid leukemia (AML) and myelofibrosis (MF). In an embodiment, the T-cell lymphoma is peripheral T-cell lymphoma. In an embodiment, the T-cell lymphoma is aggressive T-cell lymphoma. In an embodiment, the cancer is a p53 wild-type cancer. In an embodiment the small cell lung cancer is a p53 wild-type lung cancer.

In some embodiments of the use, the cancer is a B cell hematological malignancy. In some embodiments, the B cell hematological malignancy is selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, and Waldenström's macroglobulinemia (WM).

In some embodiments of the use, the cancer is a neuroendocrine cancer selected from the group consisting of pancreatic neuroendocrine cancer, carcinoid cancer, gastroenteropancreatic neuroendocrine cancer, pheochromocytoma, paraganglioma, medullary thyroid cancer, pulmonary neuroendocrine cancer, small cell lung cancer (SCLC), and thymic neuroendocrine cancer. In some embodiments of the use, the cancer is small-cell neuroendocrine prostate cancer (SCNPC). In one embodiment, the SCNPC is resistant to androgen receptor antagonists.

In some embodiments of the use, the compound of Formula (I) or Formula (II) is in a crystalline form. In some embodiments of the use, the compound of Formula (I) or Formula (II) is in a free form. In some embodiments of the use, the MDM2 inhibitor is a pharmaceutically acceptable salt of a compound of Formula (I) or Formula (II). In some embodiments of the use, the compound of Formula (I) or Formula (II) is in an amorphous form.

In some embodiments of the use, the compound of Formula (I) or Formula (II) is administered once daily at a dose selected from the group consisting of 15 mg, 25 mg, 30 mg, 50 mg, 60 mg, 75 mg, 90 mg, 100 mg, 120 mg, 150 mg, 175 mg, 180 mg, 200 mg, 225 mg, 240 mg, 250 mg, 275 mg, 300 mg, 325 mg, 350 mg, 360 mg, 375 mg, and 480 mg. In some embodiments of the use, the compound of Formula (I) or Formula (II) is administered twice daily at a dose selected from the group consisting of 15 mg, 25 mg, 30 mg, 50 mg, 60 mg, 75 mg, 90 mg, 100 mg, 120 mg, 150 mg, 175 mg, 180 mg, 200 mg, 225 mg, 240 mg, 250 mg, 275 mg, 300 mg, 325 mg, 350 mg, 360 mg, 375 mg, and 480 mg. In an embodiment of the use, the therapeutically effective amount of the MDM2 inhibitor is 120 mg. In some embodiments of the use, the human subject is treated with the MDM2 inhibitor on days 1-7 of 21-day cycle, wherein on days 8-21 the human is not treated with the MDM2 inhibitor. In some embodiments of the use, the compound of Formula (I) or Formula (II) is orally administered.

In some embodiments of the use, the human subject is previously treated with immunotherapy. In some embodiments, the immunotherapy is an ex vivo cell therapy selected from the group consisting of tumor-infiltrating lymphocytes (TILs), T-cell receptor (TCR)-engineered peripheral blood lymphocytes (PBL) and chimeric antigen receptor ((CAR)-engineered PBL). In some embodiments, the immunotherapy is an immune checkpoint protein inhibitor therapy. In some embodiments, the immune checkpoint protein inhibitor is an anti-PD-L1 antibody selected from the group consisting of BMS-936559, durvalumab, atezolizumab, avelumab, MPDL3280A, MEDI4736, MSB0010718C, MDX1105-01, and fragments, conjugates, biosimilars, or variants thereof. In some embodiments, the immune checkpoint protein inhibitor is an anti-PD-1 antibody selected from group consisting of nivolumab, tslelizimab, dostarlimab, pembrolizumab, pidilizumab, cemiplimab-rwlc, AMP-224, AMP-514, PDR001, and fragments, conjugates, biosimilars, or variants thereof. In some embodiments, the immune checkpoint protein inhibitor is an anti-CTLA-4 antibody selected from the group consisting of ipilimumab, tremelimumab, and fragments, conjugates, biosimilars, or variants thereof. In some embodiments, the immunotherapy is a T-cell engager selected from catumaxomab, FBTA05, Ertumaxomab, Ektomun, blinatumomab, solitomab, and fragments, conjugates, biosimilars, or variants thereof.

In an embodiment of the use, the human subject has Ki-67 protein proliferation rate more than 10%. In an embodiment of the method, the human subject has Ki-67 protein proliferation rate more than 15%. In an embodiment of the method, the human subject has Ki-67 protein proliferation rate more than 20%. In an embodiment of the method, the human subject has Ki-67 protein proliferation rate more than 25%.

### BRIEF DESCRIPTION OF THE DRAWINGS

The foregoing summary, as well as the following detailed description of the invention, will be better understood when read in conjunction with the appended drawings.

FIG. 1 illustrates Bcl-2 expressed in a wide range of tumor histotypes, with different extent regarding mRNA and protein levels.

FIG. 2 illustrates Bcl-XL expressed in a wide range of tumor histotypes, with different extent regarding mRNA and protein levels.

FIG. 3 illustrates Mcl-1 expressed in a wide range of tumor histotypes, with different extent regarding mRNA and protein levels.

FIG. 4 illustrates protein expression of p53, and both anti-apoptotic and pro-apoptotic proteins in CLL tumor cells following ex vivo treatment with increasing concentration of the compound of Formula (I).

FIG. 5 illustrates treatment of p53 wild-type and p53 mutant tumor cell lines with increasing concentrations of the compound of Formula (I).

FIG. 6 illustrates expression of Bak, Bax and BIM following treatment of the UKE-1 myelofibrosis cell line with the compound of Formula (I)(upper) or control (lower) for different durations.

FIG. 7 illustrates (A) gene expression analyses in PBMC samples from R/R myelofibrosis patient treated with with the compound of Formula (I); (B) gene expression analyses in PBMC samples from R/R AML patients treated with the compound of Formula (I).

FIG. 8 illustrates induced expression of Bcl-2 genes in PBMC following treatment of AML patients with the compound of Formula (I).

### DETAILED DESCRIPTION OF THE INVENTION

While preferred embodiments of the invention are shown and described herein, such embodiments are provided by way of example only and are not intended to otherwise limit the scope of the invention. Various alternatives to the described embodiments of the invention may be employed in practicing the invention.

Unless defined otherwise, all technical and scientific terms used herein have the same meaning as is commonly understood by one of skill in the art to which this invention belongs.

The terms “administered in combination with” and “co-administration” as used herein, encompass administration of two or more active pharmaceutical ingredients to a human subject so that both agents and/or their metabolites are present in the subject at the same time. Co-administration includes simultaneous administration in separate compositions, administration at different times in separate compositions, or administration in a composition in which two or more agents are present.

The term “effective amount” or “therapeutically effective amount” refers to that amount of an active pharmaceutical ingredient or combination of active pharmaceutical ingredients as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, and other factors which can readily be determined by one of ordinary skill in the art. The term also applies to a dose that will induce a particular response in target cells, (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

The term “IC₅₀” refers to the half maximal inhibitory concentration, i.e. inhibition of 50% of the desired activity. The term “EC₅₀” refers to the drug concentration at which one-half the maximum response is achieved.

“Pharmaceutically acceptable carrier” or “pharmaceutically acceptable excipient” is intended to include any and all solvents, dispersion media, coatings, antibacterial and antifungal agents, isotonic, and absorption delaying agents. The use of such media and agents for active pharmaceutical ingredients is well known in the art. Except insofar as any conventional media or agent is incompatible with the active pharmaceutical ingredient, its use in the therapeutic compositions of the invention is contemplated. Supplementary active ingredients can also be incorporated into the described compositions.

The term “pharmaceutically acceptable salt” refers to salts derived from a variety of organic and inorganic counter ions known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid and phosphoric acid. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In selected embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve proton transfer between the cocrystal and the drug, and instead involves intermolecular interactions, such as hydrogen bonding, aromatic ring stacking, or dispersive forces, between the cocrystal former and the drug in the crystal structure.

The terms “QD,” “qd,” or “q.d.” means quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quaterin die, four times a day, or four times daily.

“Solvate” refers to a compound in physical association with one or more molecules of a pharmaceutically acceptable solvent.

A “therapeutic effect” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “wild-type” refers to a gene or gene product that has the characteristics of that gene or gene product when isolated from a naturally-occurring source. A wild-type gene or gene product (e.g., a polypeptide) is that which is most frequently observed in a population and is thus arbitrarily designed the “normal” or “wild-type” form of the gene.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary from, for example, between 1% and 15% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

Compounds of the invention also include crystalline and amorphous forms of the compound of Formula (I) or Formula (II), including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

### Methods of Treating a Cancer Overexpressing One or More Bcl-2 Family Proteins

The Bcl-2 family proteins regulate mitochondrial apoptosis by modulating outer mitochondrial membrane permeability and cytochrome c release. The Bcl-2 family proteins are divided into three groups based on their primary function (1) anti-apoptotic proteins (Bcl-2, Bcl-XL, Bcl-W, Mcl-1, Bfl-1/A-1, and Bcl-B.), (2) pro-apoptotic pore-formers (Bax, Bak, BOK) and (3) pro-apoptotic BH3-only proteins (BAD, BID, BIK, BIM, BMF, HRK, NOXA, and PUMA). The Bcl-2 protein family maintains the balance between cell survival and death via sophisticated interactions between pro-apoptotic and anti-apoptotic subfamily members. The pro-apoptotic Bcl-2 family effectors, Bax and Bak, undergo oligomerization to form lipid pores, which induces the outer mitochondrial membrane permeabilization and cytochrome c release, thus inducing caspase-dependent cellular apoptosis. Antiapoptotic Bcl-2 family proteins perform their pro-survival function by binding and sequestering the pro-apoptotic effector proteins Bax and Bak and inhibiting membrane pore formation (Kang, Clin Cancer Res 2009; 15:1126-1132).

p53 can play a dual role in apoptosis in different cellular compartments. Within the nucleus, p53 acts as a transcriptional activator and induces target gene expression through its interaction with the basic transcriptional machinery components, such as transcriptional coactivator CBP/p300, eventually leading to transcription-dependent apoptosis via activation of Puma, Noxa and/or BAX. In the mitochondria, p53 interacts with anti-apoptotic Bcl-2 family proteins (Bcl-2, Bcl-XL, Bcl-W, Mcl-1, Bfl-1/A-1, and Bcl-B.), thereby triggering transcription-independent apoptosis. These binding events with different partners, occurring in distinct cellular compartments, are governed by the same binding motif of p53TAD (Tomita, WT p53, but not tumor-derived mutants, bind to Bcl2 via the DNA binding domain and induce mitochondrial permeabilization. J. Biol. Chem. 2006, 281, 8600-8606; Chi, Structural insights into the transcription-independent apoptotic pathway of p53. BMB Rep. 2014, 47(3), 167-172).

Therefore, strategies to induce p53 activation in tumors that retain wild-type p53 are promising for cancer therapy. A MDM2 inhibitor that competitively binds to its p53-binding pocket thereby leads to non-genotoxic p53 stabilization and activation of growth arrest and apoptosis pathways. In addition to nuclear stabilization, a MDM2 inhibitor causes cytoplasmic p53 accumulation and translocation to mitochondria, where p53 interacts anti-apoptotic Bcl-2 family proteins (Bcl-2, Bcl-XL, Bcl-W, Mcl-1, Bfl-1/A-1, and Bcl-B.), thereby triggering transcription-independent apoptosis (Vaseva, The transcription-independent mitochondrial p53 program is a major contributor to nutlin-induced apoptosis in tumor cells, Cell Cycle, 2009, 8:11, 1711-1719).

Bcl-2 modulates in vitro and in vivo tumor migration, invasion, autophagy and angiogenesis, promotes a cancer stem-like cell phenotype, regulates the expression of microRNA and the activity of several transcription factors and their specific target genes, controls an interleukin-1-driven axis of macrophage diversion that establishes tumor microenvironmental conditions favoring melanoma development, and is involved in mitochondrial mRNA homeostasis (D'Aguanno, Inhibition of Anti-Apoptotic Bcl-2 Proteins in Preclinical and Clinical Studies: Current Overview in Cancer, Cells, 2020, 9, 1287). Bcl-2 expression has been reported in many different tumor histotypes, including glioma, thyroid cancer, lung cancer, colorectal cancer, liver cancer, pancreatic cancer, renal cancer, breast cancer, cervical cancer, endometrial cancer, ovarian cancer, melanoma, skin cancer, and lymphoma.

Bcl-XL is responsible for the alternative splicing, including cellular stress, DNA damage, protein synthesis stalling and protein kinase C inhibition (D'Aguanno, Inhibition of Anti-Apoptotic Bcl-2 Proteins in Preclinical and Clinical Studies: Current Overview in Cancer, Cells, 2020, 9, 1287). Bcl-XL expression has been reported in many different tumor histotypes, including glioma, thyroid cancer, lung cancer, colorectal cancer, head and neck cancer, stomach cancer, liver cancer, pancreatic cancer, renal cancer, urothelial cancer, breast cancer, cervical cancer, endometrial cancer, ovarian cancer, melanoma, and lymphoma.

Mcl-1 functions at the mitochondrial outer membrane, where it neutralizes pro-apoptotic proteins such as NOXA, PUMA, BIM, and BAK. MCL-1 overexpression has been linked to the pathogenesis of multiple myeloma (Derenne, Antisense strategy shows that Mcl-1 rather than Bcl-2 or Bcl-x(L) is an essential survival protein of human myeloma cells. Blood, 2002, 100:194-9; Zhang, Myeloid cell factor-1 is a critical survival factor for multiple myeloma. Blood, 2002, 99:1885-93), chemoresistance in acute myeloid leukemia cells (Konopleva, Mechanisms of apoptosis sensitivity and resistance to the BH3 mimetic ABT-737 in acute myeloid leukemia. Cancer Cell, 2006, 100:375-88), and high tumor grade and poor prognosis in breast cancer (Ding, Myeloid cell leukemia-i inversely correlates with glycogen synthase kinase-3 beta activity and associates with poor prognosis in human breast cancer, Cancer Res. 2007, 67:4564-71). High levels of Mcl-1 have been reported in hematological malignancies and subsequently in a wide range of solid tumors, including breast, ovarian, prostate, pancreatic and non-small cell lung (NSCLC) carcinoma. Mcl-1 amplification and overexpression are also frequently associated with poor prognosis and resistance to anticancer drugs (D'Aguanno, Inhibition of Anti-Apoptotic Bcl-2 Proteins in Preclinical and Clinical Studies: Current Overview in Cancer, Cells, 2020, 9, 1287). Mcl-1 expression has been reported in many different tumor histotypes, including glioma, thyroid cancer, lung cancer, colorectal cancer, head and neck cancer, stomach cancer, liver cancer, carcinoids, pancreatic cancer, renal cancer, urothelial cancer, prostate cancer, testis cancer, breast cancer, cervical cancer, endometrial cancer, ovarian cancer, melanoma, skin cancer, and lymphoma.

Bcl-w is an anti-apoptotic protein that shares a sequence similarity with Bcl-XL, and exhibits a high conformational flexibility. BCL-w level is controlled by a number of signaling pathways, and the repertoire of transcriptional regulators largely depends on the cellular and developmental context. Increased levels of Bcl-w might be a consequence of abnormal activation of signaling cascades involved in the regulation of Bcl-w expression. A high BCL-w level can be therapeutically relevant in neurodegenerative disorders, neuron dysfunctions and after small intestinal resection, whereas BCL-w inhibition can be beneficial for cancer patients. Bcl-w expression has been reported in many different tumor histotypes, including cervical cancer, osteosarcoma, leiomyosarcomas, prostate cancer, ovarian cancer, hepatocellular carcinoma, Burkitt lymphoma, diffuse large B-cell lymphoma (DLBCL), sarcoma, B-cell chronic lymphocytic leukemia (B-CLL), and chronic myeloid leukemia (Hartman, BCL-w: apoptotic and non-apoptotic role in health and disease, Cell Death and Disease, 2020, 11:260).

Bcl-B contains conserved BH1, BH2, BH3-like, and BH4 domains, as well as a COOH terminal transmembrane domain, typical of antiapoptotic Bcl-2 family proteins that target intracellular membranes of mitochondria. From a correlative stand-point, Bcl-B expression is associated with variables of poor prognosis in breast and colorectal cancer, greater incidence of death from prostate cancer, and shorter survival as well as increased relative risk of death from small cell lung cancer (SCLC). Bcl-B is also overexpressed in many gastric cancers (Krajewska, Clin Cancer Res. 2008, 14(10), 3011-3012).

Bfl-1 is a clinically relevant cell survival factor in ˜30% of human melanomas, including those with BRAF resistance mutations; in addition, Bfl-1 upregulation confers resistance to selective BRAF inhibition. Leukemia and lymphoma is inversely correlated with cellular levels of Bfl-1, and long-term treatment with BCL-2 inhibitors and other chemotherapeutics can further upregulate the expression of BFL-1, leading to chemoresistance (Guerra, Precision targeting of Bfl-1/A1 and an atm co-dependency in human cancer, Cell Rep. 2018, 24(13), 3393-3403).

The present invention relates to a method of treating a cancer overexpressing one or more Bcl-2 anti-apoptotic/pro-survival family proteins comprising the step of administering to a human subject in need thereof a Mouse double minute 2 homolog (MDM2) inhibitor, or a pharmaceutically acceptable salt thereof. The MDM2 inhibitor causes cytoplasmic p53 accumulation and translocation to mitochondria, where p53 interacts with anti-apoptotic Bcl-2 proteins including but not limited to Bcl-2, Bcl-XL, Bcl-W, Mcl-1, Bfl-1/A-1, or Bcl-B, thereby triggering transcription-independent apoptosis. Also, the MDM2 inhibitor blocks the binding between MDM2 and p53 within the nucleus, thereby triggering transcription-dependent apoptosis by transactivation/expression of pro-apoptotic Bcl-2 family members Puma, Noxa, and Bax.

The term “cancer overexpressing one or more Bcl-2 anti-apoptotic/pro-survival family proteins” refers to a cancer characterized by overexpression of one or more Bcl-2 anti-apoptotic/pro-survival proteins selected from the group consisting of Bcl-2, Bcl-XL, Bcl-W, Mcl-1, Bfl-1/A1, and Bcl-B. As used herein, “cancer overexpressing one or more Bcl-2 family proteins” is synonymous with “Bcl-2 dependent cancer” or “Bcl-2 expression cancer” or “Bcl-2 driven cancer” and the terms are used interchangeably.

In some embodiments, the cancer overexpressing one or more Bcl-2 family proteins is characterized by overexpression of Bcl-2. In some embodiments, the cancer overexpressing one or more Bcl-2 family proteins is characterized by overexpression of Bcl-XL. In some embodiments, the cancer overexpressing one or more Bcl-2 family proteins is characterized by overexpression of Bcl-2-associated death promoter (BAD). In some embodiments, the cancer overexpressing one or more Bcl-2 family proteins is characterized by overexpression of Bcl-w.

In some embodiments, the cancer overexpressing one or more Bcl-2 family proteins is characterized by overexpression of Bcl-2, but not BAD. In some embodiments, the cancer overexpressing one or more Bcl-2 family proteins is characterized by overexpression of Bcl-2, but not Bcl-XL. In some embodiments, the cancer overexpressing one or more Bcl-2 family proteins is characterized by overexpression of Bcl-2, but not Bcl-w. In some embodiments, the cancer overexpressing one or more Bcl-2 family proteins is characterized by overexpression of Bcl-2, but not BAD, Bcl-XL and Bcl-w.

In some embodiments, the cancer overexpressing one or more Bcl-2 family proteins is characterized by overexpression one or more Bcl-2 family proteins selected from the group consisting of Bcl-2, Bcl-XL, Bcl-W, Mcl-1, Bfl-1/A1, and Bcl-B.

In some embodiments, the cancer overexpressing one or more Bcl-2 family proteins is also characterized by resistance to one or more antineoplastic agents selected from the group consisting of taxanes, platinum coordinator compounds, epidermal growth factor (EGF) inhibitors that are antibodies, EGF inhibitors that are small molecules, vascular endolithial growth factor (VEGF) inhibitors that are antibodies, VEGF kinase inhibitors that are small molecules, estrogen receptor antagonists or selective estrogen receptor modulators (SERMs), anti-tumor nucleoside derivatives, epothilones, topoisomerase inhibitors, vinca alkaloids, antibodies that are inhibitors of aVP3 integrins, small molecules that are inhibitors of caVP3 integrins, folate antagonists, ribonucleotide reductase inhibitors, anthracyclines, thalidomide and imatinib.

The disclosed methods can include the step of assessing or determining the expression levels of one or more Bcl-2 family proteins including but not limited to Bcl-2, Bcl-XL, Bcl-W, Mcl-1, Bfl-1/A1, and Bcl-B in order to select for patients likely to respond to treatment with a MDM2 inhibitor.

The present invention also relates to a method of treating a cancer overexpressing one or more Bcl-2 family proteins comprising the step of administering to a human subject in need thereof a composition comprising a Mouse double minute 2 homolog (MDM2) inhibitor, or a pharmaceutically acceptable salt thereof.

The present invention also relates to use of a MDM2 inhibitor or a pharmaceutically acceptable salt thereof for use in treating a cancer overexpressing one or more Bcl-2 family proteins.

The present invention also relates to a composition comprising a MDM2 inhibitor or a pharmaceutically acceptable salt thereof for use in treating a cancer overexpressing one or more Bcl-2 family proteins selected from the group consisting of Bcl-2, Bcl-XL, Bcl-W, Mcl-1, Bfl-1/A1, and Bcl-B. In an embodiment, the cancer overexpressing one or more Bcl-2 family proteins is selected from the group consisting of carcinomas such as cancer of the bladder, breast, colon, rectum, kidney, liver, lung (small cell lung cancer, and non-small-cell lung cancer), esophagus, gall-bladder, ovary, pancreas, stomach, cervix, thyroid, prostate, head and neck, and skin (including basal and squamous cell carcinoma, melanoma skin cancer, Merkel cell carcinoma, Kaposi Sarcoma, skin lymphomas); hematopoietic tumors of lymphoid lineage (including leukemia, acute lymphocytic leukemia, chronic myelogenous leukemia, acute lymphoblastic leukemia, B-cell lymphoma, T-cell-lymphoma, Hodgkin's lymphoma, non-Hodgkin's lymphoma, hairy cell lymphoma and Burkett's lymphoma); hematopoietic tumors of myeloid lineage (including acute and chronic myelogenous leukemias, myelodysplastic syndrome and promyelocytic leukemia); tumors of mesenchymal origin (including fibrosarcoma and rhabdomyosarcoma, and other sarcomas, e.g., soft tissue and bone); tumors of the central and peripheral nervous system (including astrocytoma, neuroblastoma, glioma, glioblastoma, and schwannomas); and other tumors (including melanoma, seminoma, teratocarcinoma, osteosarcoma, xenoderoma pigmentosum, keratoctanthoma, nasopharyngeal cancer, retinoblastoma, and thyroid follicular cancer). In an embodiment, the cancer overexpressing one or more Bcl-2 family proteins is a p53 wild-type cancer.

In some embodiments, the cancer overexpressing one or more Bcl-2 family proteins is a solid tumor. In some embodiments, the solid tumor is selected from the group consisting of alveolar soft part sarcoma, bladder cancer, breast cancer, colorectal (colon) cancer, Ewing's bone sarcoma, gastroenterological cancer, head and neck cancer, kidney cancer, leiomyosarcoma, lung cancer, melanoma, osteosarcoma, ovarian cancer, pancreatic cancer, prostate cancer, proximal or distal bile duct cancer, and neuroblastoma. In some embodiments, the solid tumor is prostate cancer. In some embodiments, the solid tumor is breast cancer. In some embodiments, the solid tumor is lung cancer. In some embodiments, the solid tumor is colorectal (colon) cancer. In some embodiments, the solid tumor is gastroenterological cancer. In some embodiments, the solid tumor is melanoma. In some embodiments, the solid tumor is lung cancer. In some embodiments, the solid tumor is kidney cancer. In some embodiments, the solid tumor is head and neck cancer. In some embodiments, the solid tumor is proximal or distal bile duct cancer. In some embodiments, the solid tumor is alveolar soft part sarcoma. In some embodiments, the solid tumor is Ewing's bone sarcoma. In some embodiments, the solid tumor is bladder cancer. In some embodiments, the solid tumor is ovarian cancer. In some embodiments, the solid tumor is leiomyosarcoma. In some embodiments, the solid tumor is osteosarcoma. In some embodiments, the solid tumor is neuroblastoma.

In an embodiment, the MDM2 inhibitor is selected from the group consisting of a compound of Formula (I), Formula (II), RG7388, Triptolide, HDM201, RG7112, CGM097A, CGM0970B, SJ-172550, SAR405838, MI-773, MX69, YH239-EE, R08994, Nutlin-3, Nutlin-3a, Nutlin-3b, Serdemetan, NSC59984, CHEMBL2386350, MK-8242, DS-3032, DS-3032B, APG-115, MI-1601, and pharmaceutically acceptable salts thereof. In an embodiment, the MDM2 inhibitor is selected from the group consisting of a compound of Formula (I), Formula (II), RG7388, HDM201, RG7112, CGM097A, CGM0970B, SAR405838, MK-8242, DS-3032B, APG-115, MI-1601, and pharmaceutically acceptable salts thereof.

In some embodiments, the present invention provides a method of treating a cancer overexpressing one or more Bcl-2 family proteins comprising the step of administering to a human subject in need thereof a therapeutically effective amount of a MDM2 inhibitor, wherein the MDM2 inhibitor is a compound of Formula (I) or Formula (II):

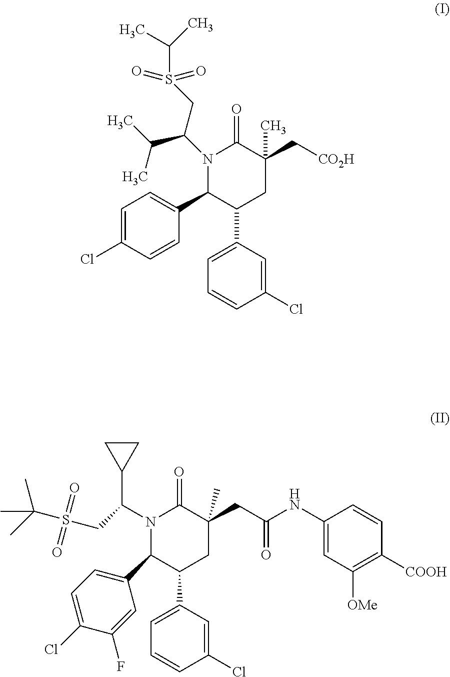 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1 ; COc1cc(NC(=O)C[C@@]2(C)C[C@H](c3cccc(Cl)c3)[C@@H](c3ccc(Cl)c(F)c3)N([C@H](CS(=O)(=O)C(C)(C)C)C3CC3)C2=O)ccc1C(=O)O]

or a pharmaceutically acceptable salt thereof.

In some embodiments, the present invention provides a method of treating a cancer overexpressing one or more Bcl-2 family proteins comprising the step of administering to a human subject in need thereof a composition comprising a therapeutically effective amount of a MDM2 inhibitor, wherein the MDM2 inhibitor is a compound of Formula (I) or Formula (II) or a pharmaceutically acceptable salt thereof.

In an embodiment, the cancer overexpressing one or more Bcl-2 family proteins is selected from the group consisting of Merkel cell carcinoma, diffuse large B-cell lymphoma (DLBCL), mantle cell lymphoma (MCL), metastatic prostate cancer, small cell lung cancer (SCLC), Burkett's lymphoma, and T-cell lymphoma.

In an embodiment, the T-cell lymphoma is indolent T-cell lymphoma.

In an embodiment, the T-cell lymphoma is aggressive T-cell lymphoma.

In an embodiment, the small cell lung cancer is a p53 wild-type lung cancer.

In an embodiment, the cancer is a B cell hematological malignancy.

In an embodiment, the B cell hematological malignancy is selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, Waldenström's macroglobulinemia (WM), acute myeloid leukemia (AML) and myelofibrosis.

In an embodiment, the human subject is previously treated with immunotherapy. In some embodiments, the cancer in the human subject is immunotherapy resistant. In an embodiment, the immunotherapy is an ex vivo cell therapy is selected from the group consisting of tumor-infiltrating lymphocytes (TILs), T-cell receptor (TCR)-engineered peripheral blood lymphocytes (PBL) and chimeric antigen receptor ((CAR)-engineered PBL).

In an embodiment, the immunotherapy is an immune checkpoint protein inhibitor therapy. In an embodiment, the immune checkpoint protein inhibitor is an anti-PD-L1 antibody selected from the group consisting of BMS-936559, durvalumab, atezolizumab, avelumab, MPDL3280A, MEDI4736, MSB0010718C, MDX1105-01, and fragments, conjugates, biosimilars, or variants thereof.

In an embodiment, the immune checkpoint protein inhibitor is an anti-PD-1 antibody selected from group consisting of nivolumab, tslelizimab, dostarlimab, pembrolizumab, pidilizumab, cemiplimab-rwlc, AMP-224, AMP-514, PDR001, and fragments, conjugates, biosimilars, or variants thereof.

In an embodiment, the immune checkpoint protein inhibitor is an anti-CTLA-4 antibody selected from the group consisting of ipilimumab, tremelimumab, and fragments, conjugates, biosimilars, or variants thereof.

In an embodiment, the immunotherapy is a T-cell engager selected from catumaxomab, FBTA05, Ertumaxomab, Ektomun, blinatumomab, solitomab, and fragments, conjugates, biosimilars, or variants thereof.

In an embodiment, the compound of Formula (I) or Formula (II) is in a crystalline form.

In an embodiment, the crystalline form is characterized by a powder X-ray diffraction pattern comprising at least three peaks at diffraction angle 2 theta degrees selected from a group consisting of peaks at approximately 11.6, 12.4, 18.6, 19.0, 21.6 and 23.6±0.1.

In an embodiment, the compound of Formula (I) or Formula (II) is in a free form.

In an embodiment, the MDM2 inhibitor is a pharmaceutically acceptable salt of a compound of Formula (I) or Formula (II).

In an embodiment, the compound of Formula (I) or Formula (II) is in an amorphous form.

In an embodiment, the compound of Formula (I) or Formula (II) is administered once daily at a dose selected from the group consisting of 15 mg, 25 mg, 30 mg, 50 mg, 60 mg, 75 mg, 90 mg, 100 mg, 120 mg, 150 mg, 175 mg, 180 mg, 200 mg, 225 mg, 240 mg, 250 mg, 275 mg, 300 mg, 325 mg, 350 mg, 360 mg, 375 mg, and 480 mg.

In an embodiment, the compound of Formula (I) or Formula (II) is administered twice daily at a dose selected from the group consisting of 15 mg, 25 mg, 30 mg, 50 mg, 60 mg, 75 mg, 90 mg, 100 mg, 120 mg, 150 mg, 175 mg, 180 mg, 200 mg, 225 mg, 240 mg, 250 mg, 275 mg, 300 mg, 325 mg, 350 mg, 360 mg, 375 mg, and 480 mg.

In an embodiment, the human is treated with the MDM2 inhibitor on days 1-7 of 21-day cycle, wherein on days 8-21 the human is not treated with the MDM2 inhibitor.

In an embodiment, the compound of Formula (I) or Formula (II) is orally administered.

In an embodiment, the therapeutically effective amount of the MDM2 inhibitor is 120 mg.

### Ki-67 Protein Analysis

Ki-67 is a protein in cells that increases as they prepare to divide into new cells. A staining process can measure the percentage of cancer cells that are positive for Ki-67. The more positive cells there are, the more quickly they are dividing and forming new cells.

In an embodiment, the human subject has Ki-67 proliferation rate more than 10%.

In an embodiment, the human subject has Ki-67 proliferation rate more than 15%.

In an embodiment, the human subject has Ki-67 proliferation rate more than 20%.

In an embodiment, the human subject has Ki-67 proliferation rate more than 25%.

In some embodiments, the cancer cells are diffuse large B cell lymphoma (DLBCL).

### MDM2 Inhibitors

The compound of Formula (I) has the structure and name shown below.

### 2-((3R,5R,6S)-5-(3-chlorophenyl)-6-(4-chlorophenyl)-1-((S)-1-(isopropylsulfonyl)-3-methylbutan-2-yl)-3-methyl-2-oxopiperidin-3-yl) acetic acid

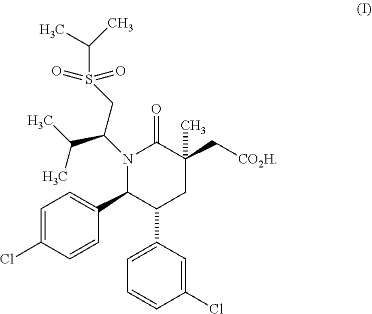 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1]

The synthesis of the compound of Formula (I) is set forth in International Applications: WO2011/153509 and WO2014/200937; U.S. Pat. Nos. 8,569,341; 9,593,129; 9,296,736; 9,623,018; 9,757,367; 9,801,867; 9,376,386 and 9,855,259; the disclosures of which are incorporated by reference herein in its entirety.

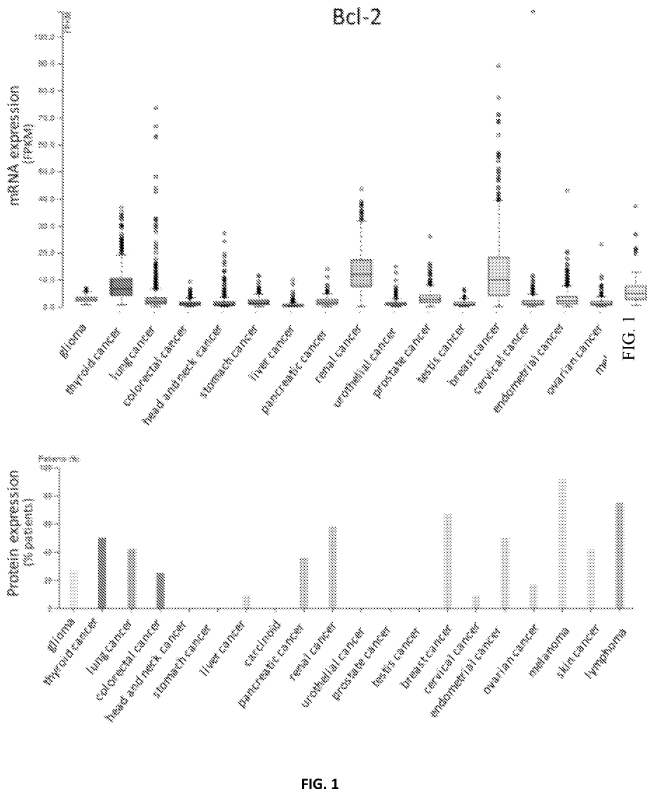

In an embodiment, the compound of Formula (I) or Formula (II) is in an amorphous form. In an embodiment, the MDM2 inhibitor is the compound of Formula (I) or Formula (II) in a crystalline form. In an embodiment, the MDM2 inhibitor is the compound of Formula (I) in a crystalline anhydrous form. In an embodiment, the MDM2 inhibitor is the compound of Formula (I) in a crystalline anhydrous form characterized by a powder X-ray diffraction pattern comprising peaks at diffraction angle 2 theta degrees at approximately 11.6, 12.4, 18.6, 19.0, 21.6 and 23.6. In an embodiment, the MDM2 inhibitor is the compound of Formula (I) in a crystalline anhydrous form having the X-ray diffraction pattern substantially shown in FIG. 1. The method of making such crystalline form was disclosed in the International Application WO2014200937, the disclosure of which is incorporated herein by reference in its entirety.

In an embodiment, the MDM2 inhibitor is a compound of Formula (II) having the structure and name shown below.

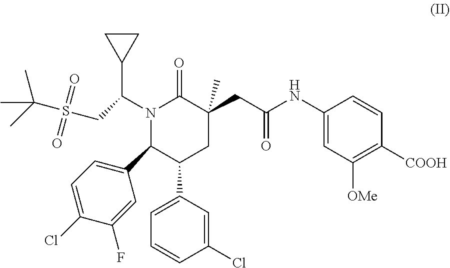 [SMILES: COc1cc(NC(=O)C[C@@]2(C)C[C@H](c3cccc(Cl)c3)[C@@H](c3ccc(Cl)c(F)c3)N([C@H](CS(=O)(=O)C(C)(C)C)C3CC3)C2=O)ccc1C(=O)O]

### 4-(2-((3R,5R,6S)-1-((S)-2-(tert-butylsulfonyl)-1-cyclopropylethyl)-6-(4-chloro-3-fluorophenyl)-5-(3-chlorophenyl)-3-methyl-2-oxopiperidin-3-yl)acetamido)-2-methoxybenzoic acid

The synthesis of the compound of Formula (II) is set forth in U.S. Pat. No. 8,952,036; the disclosure of which is incorporated by reference herein in its entirety.

### RG7388 (Idasanutlin)

In an embodiment, the MDM2 inhibitor is RG7388. RG7388 has the chemical structure and name shown as 4-[[(2R,3S,4R,5S)-3-(3-chloro-2-fluorophenyl)-4-(4-chloro-2-fluorophenyl)-4-cyano-5-(2,2-dimethylpropyl)pyrrolidine-2-carbonyl]amino]-3-methoxybenzoic acid

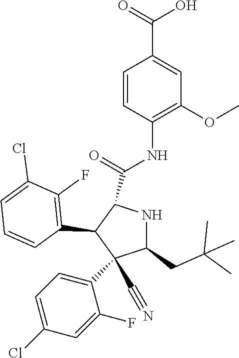 [SMILES: COc1cc(C(=O)O)ccc1NC(=O)[C@@H]1N[C@@H](CC(C)(C)C)[C@](C#N)(c2ccc(Cl)cc2F)[C@H]1c1cccc(Cl)c1F]

### Triptolide (PG490)

In an embodiment, the MDM2 inhibitor is triptolide. Triptolide has the chemical structure and name shown as (5bS,6aS,7aS,8R,8aR,9aS,9bS,10aS,10bS)-8-hydroxy-8a-isopropyl-10b-methyl-2,5,5b,6,6a,8,8a,9a,9b,10b-decahydrotris(oxireno) [2′,3′:4b,5;2″,3″:6,7;2′″,3′″:8a,9]phenanthro[1,2-c]furan-3(1H)-one

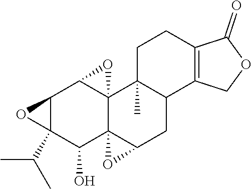

### Nutlin-3a

In an embodiment, the MDM2 inhibitor is Nutlin-3a. Nutlin-3a has the chemical structure and name shown as 4-[(4S,5R)-4,5-bis(4-chlorophenyl)-2-(4-methoxy-2-propan-2-yloxyphenyl)-4,5-dihydroimidazole-1-carbonyl]piperazin-2-one.

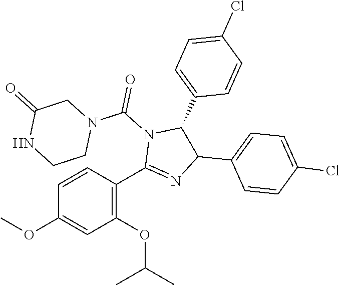 [SMILES: COc1ccc(C2=NC(c3ccc(Cl)cc3)[C@@H](c3ccc(Cl)cc3)N2C(=O)N2CCNC(=O)C2)c(OC(C)C)c1]

### HDM201

In an embodiment, the MDM2 inhibitor is HDM201. HDM201 has the chemical structure and name shown as (4S)-5-(5-chloro-1-methyl-2-oxopyridin-3-yl)-4-(4-chlorophenyl)-2-(2,4-dimethoxypyrimidin-5-yl)-3-propan-2-yl-4H-pyrrolo[3,4-d]imidazol-6-one.

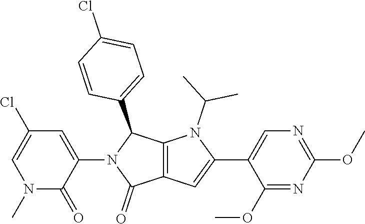 [SMILES: COc1ncc(-c2cc3c(n2C(C)C)[C@H](c2ccc(Cl)cc2)N(c2cc(Cl)cn(C)c2=O)C3=O)c(OC)n1]

### RG7112

In an embodiment, the MDM2 inhibitor is RG7112. RG7112 has the chemical structure and name shown as [(4S,5R)-2-(4-tert-butyl-2-ethoxyphenyl)-4,5-bis(4-chlorophenyl)-4,5-dimethylimidazol-1-yl]-[4-(3-methylsulfonylpropyl)piperazin-1-yl]methanone.

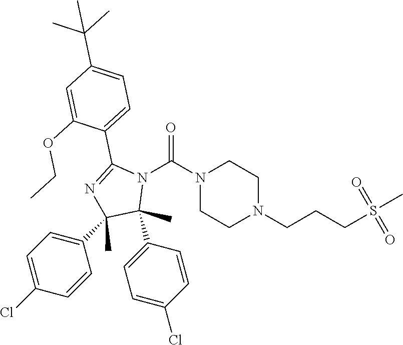 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CCCS(C)(=O)=O)CC1]

### CGM097A

In an embodiment, the MDM2 inhibitor is CGM097A. CGM097A has the chemical structure and name shown as (1S)-1-(4-chlorophenyl)-6-methoxy-2-[4-[methyl-[[4-(4-methyl-3-oxopiperazin-1-yl)cyclohexyl]methyl]amino]phenyl]-7-propan-2-yloxy-1,4-dihydroisoquinolin-3-one.

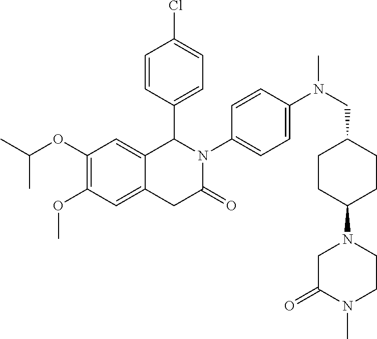 [SMILES: COc1cc2c(cc1OC(C)C)C(c1ccc(Cl)cc1)N(c1ccc(N(C)C[C@H]3CC[C@H](N4CCN(C)C(=O)C4)CC3)cc1)C(=O)C2]

### Nutlin-3

In an embodiment, the MDM2 inhibitor is nutlin-3. Nutlin-3 has the chemical structure and name shown as 4-[4,5-bis(4-chlorophenyl)-2-(4-methoxy-2-propan-2-yloxyphenyl)-4,5-dihydroimidazole-1-carbonyl]piperazin-2-one.

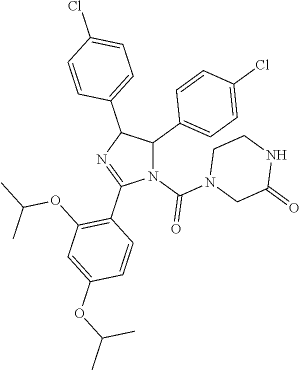 [SMILES: CC(C)Oc1ccc(C2=NC(c3ccc(Cl)cc3)C(c3ccc(Cl)cc3)N2C(=O)N2CCNC(=O)C2)c(OC(C)C)c1]

### SJ-172550

In an embodiment, the MDM2 inhibitor is SJ-172550. SJ-172550 has the chemical structure and name shown as methyl 2-[2-chloro-6-ethoxy-4-[(3-methyl-5-oxo-1-phenylpyrazol-4-ylidene)methyl]phenoxy]acetate.

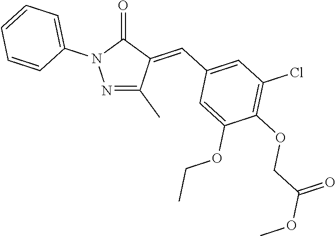 [SMILES: CCOc1cc(/C=C2/C(=O)N(c3ccccc3)N=C2C)cc(Cl)c1OCC(=O)OC]

### SAR405838 (MI-77301)

In an embodiment, the MDM2 inhibitor is SAR405838. SAR405838 has the chemical structure and name shown as (2′R,3R,3′S,5′S)-6-chloro-3′-(3-chloro-2-fluorophenyl)-5′-(2,2-dimethylpropyl)-N-(4-hydroxycyclohexyl)-2-oxospiro[1H-indole-3,4′-pyrrolidine]-2′-carboxamide.

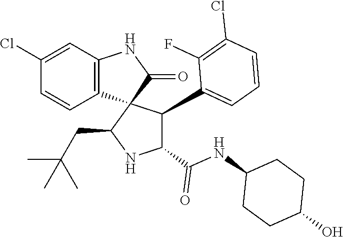 [SMILES: CC(C)(C)C[C@@H]1N[C@@H](C(=O)N[C@H]2CC[C@H](O)CC2)[C@H](c2cccc(Cl)c2F)[C@]12C(=O)Nc1cc(Cl)ccc12]

### MI-773

In an embodiment, the MDM2 inhibitor is MI-773. MI-773 has the chemical structure and name shown as (2′R,3S,3′S,5′R)-6-chloro-3′-(3-chloro-2-fluorophenyl)-5′-(2,2-dimethylpropyl)-N-(4-hydroxycyclohexyl)-2-oxospiro[1H-indole-3,4′-pyrrolidine]-2′-carboxamide.

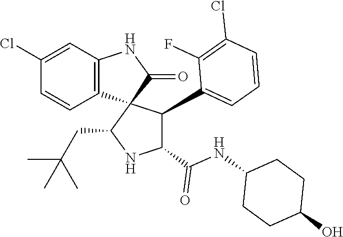 [SMILES: CC(C)(C)C[C@H]1N[C@@H](C(=O)N[C@H]2CC[C@H](O)CC2)[C@H](c2cccc(Cl)c2F)[C@]12C(=O)Nc1cc(Cl)ccc12]

### MX69

In an embodiment, the MDM2 inhibitor is MX69. MX69 has the chemical structure and name shown as 4-[8-[(3,4-dimethylphenyl)sulfamoyl]-3a,4,5,9b-tetrahydro-3H-cyclopenta[c]quinolin-4-yl]benzoic acid.

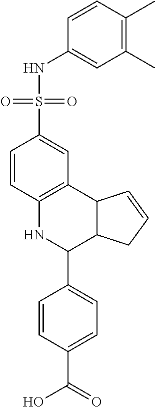 [SMILES: Cc1ccc(NS(=O)(=O)c2ccc3c(c2)C2C=CCC2C(c2ccc(C(=O)O)cc2)N3)cc1C]

### YH239-EE

In an embodiment, the MDM2 inhibitor is YH239-EE. YH239-EE has the chemical structure and name shown as ethyl 3-[2-(tert-butylamino)-1-[(4-chlorophenyl)methyl-formylamino]-2-oxoethyl]-6-chloro-1H-indole-2-carboxylate.

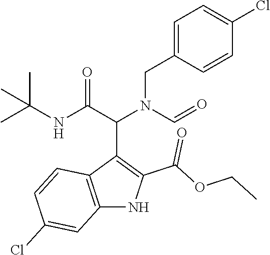 [SMILES: CCOC(=O)c1[nH]c2cc(Cl)ccc2c1C(C(=O)NC(C)(C)C)N(C=O)Cc1ccc(Cl)cc1]

### R08994

In an embodiment, the MDM2 inhibitor is R08994. R08994 has the chemical structure and name shown as (2′R,3R,3′S,5′S)—N-(4-carbamoyl-2-methoxyphenyl)-6-chloro-3′-(3-chloro-2-fluorophenyl)-5′-(2,2-dimethylpropyl)-2-oxospiro[1H-indole-3,4′-pyrrolidine]-2′-carboxamide.

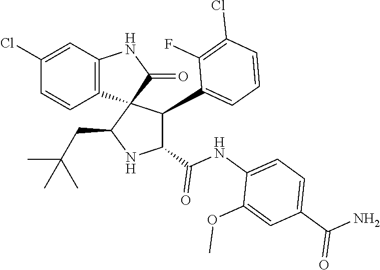 [SMILES: COc1cc(C(N)=O)ccc1NC(=O)[C@@H]1N[C@@H](CC(C)(C)C)[C@@]2(C(=O)Nc3cc(Cl)ccc32)[C@H]1c1cccc(Cl)c1F]

### Nutlin-3b

In an embodiment, the MDM2 inhibitor is nutlin-3b. Nutlin-3b has the chemical structure and name shown as 4-[(4R,5S)-4,5-bis(4-chlorophenyl)-2-(4-methoxy-2-propan-2-yloxyphenyl)-4,5-dihydroimidazole-1-carbonyl]piperazin-2-one.

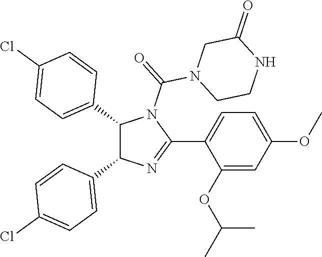 [SMILES: COc1ccc(C2=N[C@H](c3ccc(Cl)cc3)[C@H](c3ccc(Cl)cc3)N2C(=O)N2CCNC(=O)C2)c(OC(C)C)c1]

### Serdemetan (JNJ-26854165)

In an embodiment, the MDM2 inhibitor is Serdemetan. Serdemetan has the chemical structure and name shown as 1-N-[2-(1H-indol-3-yl)ethyl]-4-N-pyridin-4-ylbenzene-1,4-diamine.

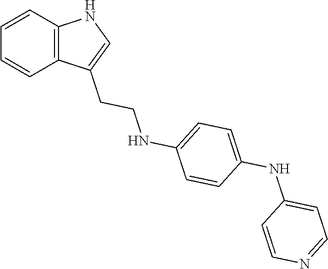 [SMILES: c1ccc2c(CCNc3ccc(Nc4ccncc4)cc3)c[nH]c2c1]

### NSC59984

In an embodiment, the MDM2 inhibitor is NSC59984. NSC59984 has the chemical structure and name shown as (E)-1-(4-methylpiperazin-1-yl)-3-(5-nitrofuran-2-yl)prop-2-en-1-one

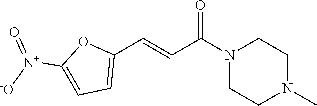

### CHEMBL2386350

In an embodiment, the MDM2 inhibitor is CHEMBL2386350. CHEMBL2386350 has the chemical structure and name shown as 2-[4-[(4S,5R)-2-(4-tert-butyl-2-ethoxyphenyl)-4,5-bis(4-chlorophenyl)-4,5-dimethylimidazole-1-carbonyl]piperazin-1-yl]-1-morpholin-4-ylethanone.

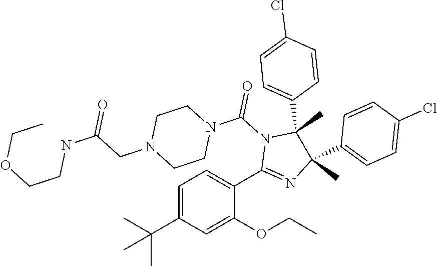 [SMILES: CCOCCNC(=O)CN1CCN(C(=O)N2C(c3ccc(C(C)(C)C)cc3OCC)=N[C@@](C)(c3ccc(Cl)cc3)[C@@]2(C)c2ccc(Cl)cc2)CC1]

### CGM0970B

In an embodiment, the MDM2 inhibitor is CGM0970B. CGM0970B has the chemical structure and name shown as (1R)-1-(4-chlorophenyl)-6-methoxy-2-[4-[methyl-[[4-(4-methyl-3-oxopiperazin-1-yl)cyclohexyl]methyl]amino]phenyl]-7-propan-2-yloxy-1,4-dihydroisoquinolin-3-one.

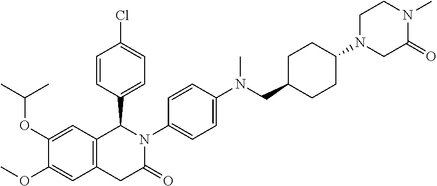 [SMILES: COc1cc2c(cc1OC(C)C)[C@@H](c1ccc(Cl)cc1)N(c1ccc(N(C)C[C@H]3CC[C@H](N4CCN(C)C(=O)C4)CC3)cc1)C(=O)C2]

### MK-8242

In an embodiment, the MDM2 inhibitor is MK-8242. MK-8242 has the chemical structure and name shown as 4-amino-1-[(2R,3S,4S,5R)-3,4-dihydroxy-5-(hydroxymethyl)oxolan-2-yl]pyrimidin-2-one.

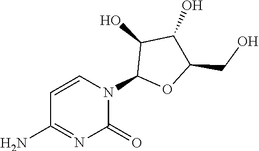 [SMILES: Nc1ccn([C@@H]2O[C@H](CO)[C@@H](O)[C@@H]2O)c(=O)n1]

### DS-3032

In an embodiment, the MDM2 inhibitor is DS-3032. DS-3032 has the chemical structure and name shown as (3′R,4'S,5′R)—N-((3R,6S)-6-carbamoyltetrahydro-2H-pyran-3-yl)-6″-chloro-4′-(2-chloro-3-fluoropyridin-4-yl)-4,4-dimethyl-2″-oxodispiro[cyclohexane-1,2′-pyrrolidine-3′,3″-indoline]-5′-carboxamide.

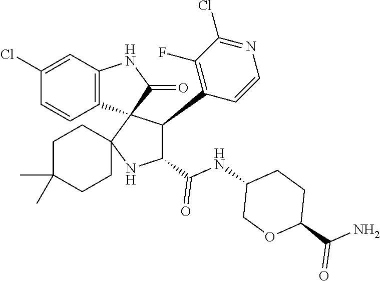 [SMILES: CC1(C)CCC2(CC1)N[C@@H](C(=O)N[C@@H]1CC[C@@H](C(N)=O)OC1)[C@H](c1ccnc(Cl)c1F)[C@]21C(=O)Nc2cc(Cl)ccc21]

### DS-3032B

In an embodiment, the MDM2 inhibitor is DS-3032B. DS-3032B has the chemical structure and name shown as (3′R,4'S,5′R)—N-((3R,6S)-6-carbamoyltetrahydro-2H-pyran-3-yl)-6″-chloro-4′-(2-chloro-3-fluoropyridin-4-yl)-4,4-dimethyl-2″-oxodispiro[cyclohexane-1,2′-pyrrolidine-3′,3″-indoline]-5′-carboxamide 4-methylbenzenesulfonate.

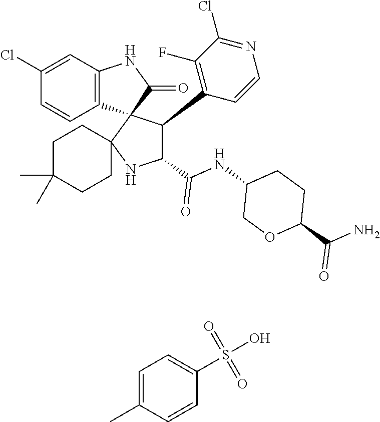 [SMILES: CC1(C)CCC2(CC1)N[C@@H](C(=O)N[C@@H]1CC[C@@H](C(N)=O)OC1)[C@H](c1ccnc(Cl)c1F)[C@]21C(=O)Nc2cc(Cl)ccc21 ; Cc1ccc(S(=O)(=O)O)cc1]

### HDM201

In an embodiment, the MDM2 inhibitor is HDM201. HDM201 has the chemical structure and name shown as (4S)-5-(5-chloro-1-methyl-2-oxopyridin-3-yl)-4-(4-chlorophenyl)-2-(2,4-dimethoxypyrimidin-5-yl)-3-propan-2-yl-4H-pyrrolo[3,4-d]imidazol-6-one.

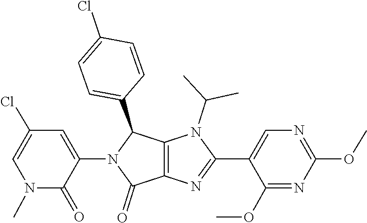 [SMILES: COc1ncc(-c2nc3c(n2C(C)C)[C@H](c2ccc(Cl)cc2)N(c2cc(Cl)cn(C)c2=O)C3=O)c(OC)n1]

### APG-115

In an embodiment, the MDM2 inhibitor is APG-115. APG-115 has the chemical structure and name shown as 4-((3′R,4'S,5′R)-6″-Chloro-4′-(3-chloro-2-fluorophenyl)-1′-ethyl-2″-oxodispiro[cyclohexane-1,2′-pyrrolidine-3′,3″-indoline]-5′-carboxamido)bicyclo[2.2.2]octane-1-carboxylic Acid.

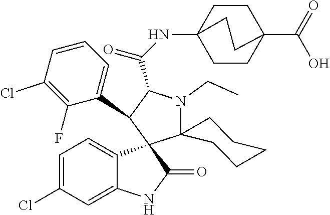 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

### MI-1061

In an embodiment, the MDM2 inhibitor is APG-115. APG-115 has the chemical structure and name shown as 4-((3′R,4'S,5′R)-6″-chloro-4′-(3-chloro-2-fluorophenyl)-2″-oxodispiro[cyclohexane-1,2′-pyrrolidine-3′,3″-indoline]-5′-carboxamido)benzoic acid.

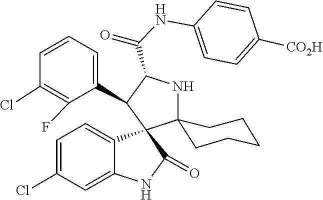 [SMILES: O=C(O)c1ccc(NC(=O)[C@@H]2NC3(CCCCC3)[C@@]3(C(=O)Nc4cc(Cl)ccc43)[C@H]2c2cccc(Cl)c2F)cc1]

### Immunotherapy

In some embodiments, the immunotherapy described herein refers to an immune checkpoint immunotherapy wherein an immune checkpoint protein inhibitor is administered to a human subject in need thereof. The immune checkpoint protein inhibitor is an agent that modulates a target selected from the group consisting of CTLA-4, PD-1, PD-L1, PD-L2, LAG3, B7-H3, B7-H4, KIR, OX40, IDO-1, IDO-2, CEACAM1, INFR5F4, BTLA, OX4OL, and TIM3 or combinations thereof.

In some embodiments, the immunotherapy is a T-cell engager.

In some embodiments, the immune checkpoint protein inhibitor is a PD-1 inhibitor selected from the group consisting of nivolumab, tslelizimab, dostarlimab, pembrolizumab, atezolizumab, avelumab, and durvalumab.

In some embodiments, the immune checkpoint protein inhibitor is a CTLA-4 inhibitor selected from the group consisting of ipilimumab and tremelimumab.

In some embodiments, the immune checkpoint protein inhibitor comprises a PD-1 immune checkpoint protein inhibitor and a CTLA-4 immune checkpoint protein inhibitor.

In some embodiments, the immune checkpoint protein inhibitor is a PD-L1 inhibitor selected from the group consisting of BMS-936559, durvalumab, atezolizumab, avelumab, MPDL3280A, MEDI4736, MSB0010718C, MDX1105-01, and fragments, conjugates, biosimilars, or variants thereof.

In an embodiment, the immune checkpoint protein inhibitor is an anti-PD-L2 antibody. In one embodiment, the anti-PD-L2 antibody is rHIgM12B7A.

### PD-1 Inhibitors

The PD-1 inhibitor may be any PD-1 inhibitor or PD-1 blocker known in the art. In particular, it is one of the PD-1 inhibitors or blockers described in more detail in the following paragraphs. The terms “inhibitor” and “blocker” are used interchangeably herein in reference to PD-1 inhibitors. For avoidance of doubt, references herein to a PD-1 inhibitor that is an antibody may refer to a compound or antigen-binding fragments, variants, conjugates, or biosimilars thereof. For avoidance of doubt, references herein to a PD-1 inhibitor may also refer to a compound or a pharmaceutically acceptable salt, ester, solvate, hydrate, cocrystal, or prodrug thereof.

In some embodiments, the compositions and methods described include a PD-1 inhibitor that binds human PD-1 with a K_{D} of about 100 pM or lower, binds human PD-1 with a K_{D} of about 90 pM or lower, binds human PD-1 with a K_{D} of about 80 pM or lower, binds human PD-1 with a K_{D} of about 70 pM or lower, binds human PD-1 with a K_{D} of about 60 pM or lower, binds human PD-1 with a K_{D} of about 50 pM or lower, binds human PD-1 with a K_{D} of about 40 pM or lower, or binds human PD-1 with a K_{D} of about 30 pM or lower.

In some embodiments, the compositions and methods described include a PD-1 inhibitor that binds to human PD-1 with a k_{assoc} of about 7.5×10⁵ l/M·s or faster, binds to human PD-1 with a k_{assoc} of about 7.5×10⁵ l/M·s or faster, binds to human PD-1 with a k_{assoc} of about 8×10⁵ l/M·s or faster, binds to human PD-1 with a k_{assoc} of about 8.5×10⁵ l/M·s or faster, binds to human PD-1 with a k_{assoc} of about 9×10⁵ l/M·s or faster, binds to human PD-1 with a k_{assoc} of about 9.5×10⁵ l/M·s or faster, or binds to human PD-1 with a k_{assoc} of about 1×10⁶ l/M·s or faster.

In some embodiments, the compositions and methods described include a PD-1 inhibitor that binds to human PD-1 with a k_{dissoc} of about 2×10⁻⁵ l/s or slower, binds to human PD-1 with a k_{dissoc} of about 2.1×10⁻⁵ l/s or slower, binds to human PD-1 with a k_{dissoc} of about 2.2×10⁻⁵ l/s or slower, binds to human PD-1 with a k_{dissoc} of about 2.3×10⁻⁵ l/s or slower, binds to human PD-1 with a k_{dissoc} of about 2.4×10-5 l/s or slower, binds to human PD-1 with a k_{dissoc} of about 2.5×10⁻⁵ l/s or slower, binds to human PD-1 with a k_{dissoc} of about 2.6×10⁻⁵ l/s or slower or binds to human PD-1 with a k_{dissoc} of about 2.7×10⁻⁵ l/s or slower, binds to human PD-1 with a k_{dissoc} of about 2.8×10⁻⁵ l/s or slower, binds to human PD-1 with a k_{dissoc} of about 2.9×10⁻⁵ l/s or slower, or binds to human PD-1 with a k_{dissoc} of about 3×10⁻⁵ l/s or slower.

In some embodiments, the compositions and methods described include a PD-1 inhibitor that blocks or inhibits binding of human PD-LI or human PD-L2 to human PD-1 with an IC₅₀ of about 10 nM or lower, blocks or inhibits binding of human PD-LI or human PD-L2 to human PD-1 with an IC₅₀ of about 9 nM or lower, blocks or inhibits binding of human PD-LI or human PD-L2 to human PD-1 with an IC₅₀ of about 8 nM or lower, blocks or inhibits binding of human PD-LI or human PD-L2 to human PD-1 with an IC₅₀ of about 7 nM or lower, blocks or inhibits binding of human PD-LI or human PD-L2 to human PD-1 with an IC₅₀ of about 6 nM or lower, blocks or inhibits binding of human PD-LI or human PD-L2 to human PD-1 with an IC₅₀ of about 5 nM or lower, blocks or inhibits binding of human PD-LI or human PD-L2 to human PD-1 with an IC₅₀ of about 4 nM or lower, blocks or inhibits binding of human PD-LI or human PD-L2 to human PD-1 with an IC₅₀ of about 3 nM or lower, blocks or inhibits binding of human PD-LI or human PD-L2 to human PD-1 with an IC₅₀ of about 2 nM or lower, or blocks or inhibits binding of human PD-LI or human PD-L2 to human PD-1 with an IC₅₀ of about 1 nM or lower.

In an embodiment, an anti-PD-1 antibody comprises nivolumab (Bristol-Myers Squibb) or antigen-binding fragments, conjugates, or variants thereof. Nivolumab is referred to as 5C4 in International Patent Publication No. WO 2006/121168. Nivolumab is assigned CAS registry number 946414-94-4 and is also known to those of ordinary skill in the art as BMS-936558, MDX-1106 or ONO-4538. Nivolumab is a fully human IgG4 antibody blocking the PD-1 receptor.

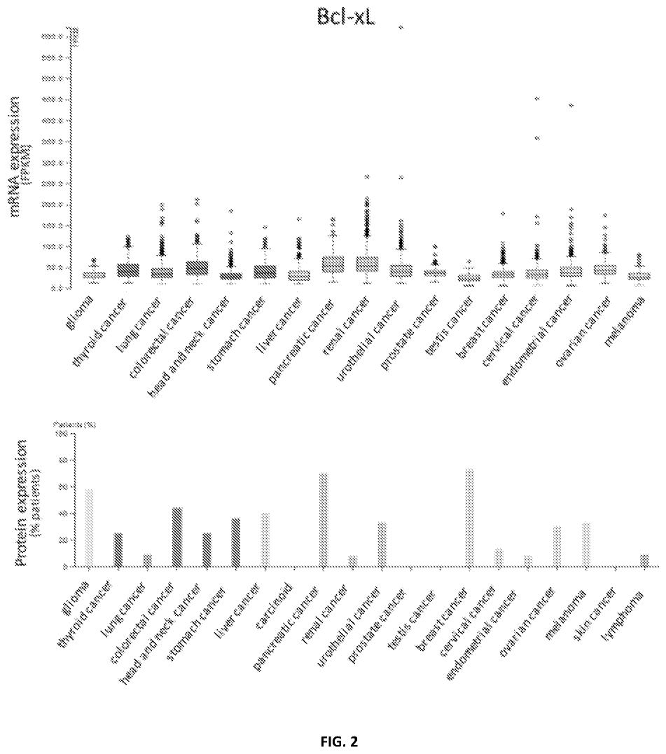

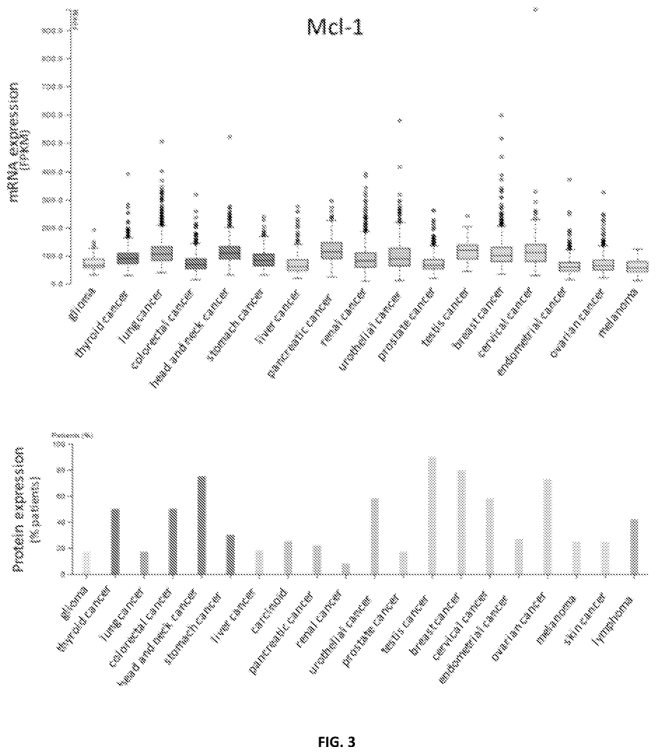

In an embodiment, the anti-PD-1 antibody is an antibody disclosed and/or prepared according to U.S. Pat. No. 8,008,449 or U.S. Patent Application Publication No. 2009/0217401 or 2013/0133091, the disclosures of which are specifically incorporated by reference herein. For example, in an embodiment, the monoclonal antibody includes 5C4 (referred to herein as nivolumab), 17D8, 2D3, 4H1, 4A11, 7D3, and 5F4, described in U.S. Pat. No. 8,008,449, the disclosures of which are hereby incorporated by reference. The PD-1 antibodies 17D8, 2D3, 4H1, 5C4, and 4A11, are all directed against human PD-1, bind specifically to PD-1 and do not bind to other members of the CD28 family. The sequences and CDR regions for these antibodies are provided in U.S. Pat. No. 8,008,449, in particular FIG. 1 through FIG. 12; all of which are incorporated by reference herein in their entireties.

In another embodiment, the anti-PD-1 antibody comprises pembrolizumab, which is commercially available from Merck, or antigen-binding fragments, conjugates, or variants thereof. Pembrolizumab is referred to as h409AI I in International Patent Publication No. WO 2008/156712, U.S. Pat. No. 8,354,509 and U.S. Patent Application Publication No. 2010/0266617, 2013/0108651 and 2013/0109843. Pembrolizumab has an immunoglobulin G4, anti-(human protein PDCD1 (programmed cell death 1)) (human-Mus musculus monoclonal heavy chain), disulfide with human-Mus musculus monoclonal light chain, dimer structure. The structure of pembrolizumab may also be described as immunoglobulin G4, anti-(human programmed cell death 1); humanized mouse monoclonal [228-L-proline(H10-S>P)]γ4 heavy chain (134-218′)-disulfide with humanized mouse monoclonal κ light chain dimer (226-226″:229-229″)-bisdisulfide. Pembrolizumab is assigned CAS registry number 1374853-91-4 and is also known as lambrolizumab, MK-3475, and SCH-900475.

In an embodiment, the anti-PD-1 antibody is an antibody disclosed in U.S. Pat. No. 8,354,509 the disclosure of which are specifically incorporated by reference herein.

In an embodiment, the anti-PD-1 antibody is pidilizumab, which is also known as CT-011 (CureTech), and which is disclosed in U.S. Pat. No. 8,686,119 B2, the disclosures of which are specifically incorporated by reference herein.

In another embodiment, anti-PD-1 antibodies and other PD-1 inhibitors include those described in U.S. Pat. Nos. 8,287,856, 8,580,247 and 8,168,757, the disclosures of which are hereby incorporated by reference. In another embodiment, antibodies that compete with any of these antibodies for binding to PD-1 are also included. In another embodiment, the anti-PD-1 antibody is an antibody disclosed in U.S. Pat. No. 8,735,553 the disclosure of which are incorporated herein by reference.

The PD-1 inhibitor may also be a small molecule or peptide, or a peptide derivative, such as those described in U.S. Pat. Nos. 8,907,053; 9,096,642 and 9,044,442; 1,2,4 oxadiazole compounds and derivatives such as those described in U.S. Patent Application Publication No. 2015/0073024; cyclic peptidomimetic compounds and derivatives such as those described in U.S. Patent Application Publication No. 2015/0073042; cyclic compounds and derivatives such as those described in U.S. Patent Application Publication No. 2015/0125491; 1,3,4 oxadiazole and 1,3,4 thiadiazole compounds and derivatives such as those described in International Patent Application Publication No. WO 2015/033301; peptide-based compounds and derivatives such as those described in International Patent Application Publication No. WO 2015/036927 and WO 2015/04490, or a macrocyclic peptide-based compounds and derivatives such as those described in U.S. Patent Application Publication No. 2014/0294898; the disclosures of each of which are hereby incorporated by reference in their entireties.

In an embodiment, the PD-1 inhibitor is selected from group consisting of nivolumab, tslelizimab, dostarlimab, pembrolizumab, pidilizumab, AMP-224, AMP-514, PDR001, AUNP-12 and combinations thereof. In an embodiment the PD-1 inhibitor is nivolumab. In an embodiment the PD-1 inhibitor is pembrolizumab. In an embodiment the PD-1 inhibitor is Pidilizumab. In an embodiment the PD-1 inhibitor is AMP-224.

### PD-L1 and PD-L2 Inhibitors

The PD-L1 or PD-L2 inhibitor may be any PD-L1 or PD-L2 inhibitor or blocker known in the art. In particular, it is one of the PD-L1 or PD-L2 inhibitors or blockers described in more detail in the following paragraphs. The terms “inhibitor” and “blocker” are used interchangeably herein in reference to PD-L1 and PD-L2 inhibitors. For avoidance of doubt, references herein to a PD-L1 or PD-L2 inhibitor that is an antibody may refer to a compound or antigen-binding fragments, variants, conjugates, or biosimilars thereof. For avoidance of doubt, references herein to a PD-L1 or PD-L2 inhibitor may refer to a compound or a pharmaceutically acceptable salt, ester, solvate, hydrate, cocrystal, or prodrug thereof.

In an embodiment, the anti-PD-L1 antibody is durvalumab, which is also known as MEDI4736 (Medimmune) or antigen-binding fragments, conjugates, or variants thereof. In an embodiment, the anti-PD-L1 antibody is an antibody disclosed in U.S. Pat. No. 8,779,108 or U.S. Patent Application Publication No. 2013/0034559, the disclosures of which are specifically incorporated by reference herein.

In an embodiment, the anti-PD-L1 antibody is atezolizumab, also known as MPDL3280A or RG7446 (Genentech) or antigen-binding fragments, conjugates, or variants thereof. In an embodiment, the anti-PD-L1 antibody is an antibody disclosed in U.S. Pat. No. 8,217,149, the disclosure of which is specifically incorporated by reference herein. In an embodiment, the anti-PD-L1 antibody is an antibody disclosed in U.S. Patent Application Publication No. 2010/0203056, the disclosure of which is specifically incorporated by reference herein.

In an embodiment, the anti-PD-L1 antibody is avelumab, also known as MSB0010718C (Merck KGaA/EMD Serono) or antigen-binding fragments, conjugates, or variants thereof. In an embodiment, the anti-PD-L1 antibody is an antibody disclosed in U.S. Patent Application Publication No. 2014/0341917, the disclosure of which is specifically incorporated by reference herein.

In an embodiment, the anti-PD-L1 antibody is MDX-1105, also known as BMS-935559, which is disclosed in U.S. Pat. No. 7,943,743, the disclosures of which are specifically incorporated by reference herein. In an embodiment, the anti-PD-L1 antibody is selected from the anti-PD-L1 antibodies disclosed in U.S. Pat. No. 7,943,743 which is specifically incorporated by reference herein.

In an embodiment, the anti-PD-L1 antibody is a commercially-available monoclonal antibody, such as INVIVOMAB anti-m-PD-L1 clone 10F.9G2 (BioXCell). A number of commercially-available anti-PD-L1 antibodies are known to one of ordinary skill in the art.

In an embodiment, the anti-PD-L2 antibody is a commercially-available monoclonal antibody, such as BIOLEGEND 24F.10C12 Mouse IgG2a, K isotype (Biolegend), anti-PD-L2 antibody (Sigma-Aldrich), or other commercially-available anti-PD-L2 antibodies known to one of ordinary skill in the art.

In an embodiment, the PD-L1 inhibitor is an anti-PD-L1 antibody. In an embodiment, the PD-L1 inhibitor is selected from the group consisting of Atezolizumab, Avelumab, Durvalumab, BMS-936559 and combinations thereof. In an embodiment, the anti-PD-L1 inhibitor is durvalumab (MEDI4736). In an embodiment, the anti-PD-L1 inhibitor is BMS-936559 (also known as MDX-1105-01). In an embodiment, the anti-PD-L1 inhibitor is Atezolizumab. In an embodiment, the anti-PD-L1 inhibitor is Avelumab.

In an embodiment, the immunotherapy is a PD-L2 inhibitor. In an embodiment, the PD-L2 inhibitor is an anti-PD-L2 antibody. In one embodiment, the anti-PD-L2 antibody is rHIgM12B7A.

### CTLA-4 Inhibitors

In some embodiments, the at least one immune checkpoint protein inhibitor is an inhibitor of CTLA-4. In some embodiments, the at least one immune checkpoint protein inhibitor is an antibody against CTLA-4. In some embodiments, the at least one immune checkpoint protein inhibitor is a monoclonal antibody against CTLA-4. In other or additional embodiments, the at least one immune checkpoint protein inhibitor is a human or humanized antibody against CTLA-4. In one embodiment, the anti-CTLA-4 antibody blocks the binding of CTLA-4 to CD80 (B7-1) and/or CD86 (B7-2) expressed on antigen presenting cells. Exemplary antibodies against CTLA-4 include: Bristol Meyers Squibb's anti-CTLA-4 antibody ipilimumab (also known as Yervoy™, MDX-010, BMS-734016 and MDX-101); anti-CTLA4 Antibody, clone 9H10 from Millipore; Pfizer's tremelimumab (CP-675,206, ticilimumab); and anti-CTLA4 antibody clone BNI3 from Abcam.

In some embodiments, the anti-CTLA-4 antibody is an anti-CTLA-4 antibody disclosed in any of the following patent publications (which is incorporated by reference in its entirety): WO 2001014424; WO 2004035607; US2005/0201994; EP 1212422; WO 2003086459; WO 2012120125; WO 2000037504; WO 2009100140; WO 200609649; WO 2005092380; WO 2007123737; WO 2006029219; WO20100979597; WO200612168 and WO1997020574. Additional CTLA-4 antibodies are described in U.S. Pat. Nos. 5,811,097, 5,855,887, 6,051,227, and 6,984,720; in PCT Publication No. WO 200114424 and WO 200037504; and in U.S. Publication No. 2002/0039581 and 2002/086014; and/or U.S. Pat. Nos. 5,977,318, 6,682,736, 7, 109,003 and 7,132,281, incorporated herein by reference).

In some embodiments, the CTLA-4 inhibitor is a CTLA-4 ligand as disclosed in WO 1996040915. In other embodiments the CTLA-4 inhibitor may be B7-like peptides or nucleic acid molecules disclosed in U.S. Pat. No. 6,630,575.

### T-Cell Engager

In an embodiment, the immunotherapy is a T-cell engager. In some embodiments, the T cell engager is selected from an antigen binding domain or ligand that binds to (e.g., and in some embodiments activates) one or more of CD3, TCRa, TCRp, TCRy, TCRC, ICOS, CD28, CD27, HVEM, LIGHT, CD40, 4-1BB, OX40, DR3, GITR, CD30, TIM1, SLAM, CD2, or CD226. In other embodiments, the T cell engager is selected from an antigen binding domain or ligand that binds to and does not activate one or more of CD3, TCRa, TCRp, TCRy, TCRC, ICOS, CD28, CD27, HVEM, LIGHT, CD40, 4-1BB, OX40, DR3, GITR, CD30, TIM1, SLAM, CD2, or CD226. In some embodiments, the T cell engager binds to CD3.

In some embodiments, the T cell engager is selected from the group consisting of: Catumaxomab mAb (anti CD3× anti-EpCAM), FBTA05/Lymphomun (CD3× anti-anti-CD20), duly cable Eritrea mAb (Ertumaxomab) (anti-CD3×anti-HER2/neu), Ektomun (anti-CD3×anti-GD2), Bona spit mAb (blinatumomab) and B. thuringiensis FIG mAb (solitomab).

### Pharmaceutical Compositions

In some embodiments, the invention provides pharmaceutical compositions comprising a MDM2 inhibitor or a pharmaceutically acceptable salt thereof for treating a cancer overexpressing one or more Bcl-2 family proteins, wherein the cancer overexpressing one or more Bcl-2 family proteins is selected from the group consisting of carcinomas such as cancer of the bladder, breast, colon, rectum, kidney, liver, lung (small cell lung cancer, and non-small-cell lung cancer), esophagus, gall-bladder, ovary, pancreas, stomach, cervix, thyroid, prostate, head and neck, and skin (including basal and squamous cell carcinoma, melanoma skin cancer, Merkel cell carcinoma, Kaposi Sarcoma, skin lymphomas); hematopoietic tumors of lymphoid lineage (including leukemia, acute lymphocytic leukemia, chronic myelogenous leukemia, acute lymphoblastic leukemia, B-cell lymphoma, T-cell-lymphoma, Hodgkin's lymphoma, non-Hodgkin's lymphoma, hairy cell lymphoma and Burkett's lymphoma); hematopoietic tumors of myeloid lineage (including acute and chronic myelogenous leukemias, myelodysplastic syndrome and promyelocytic leukemia); tumors of mesenchymal origin (including fibrosarcoma and rhabdomyosarcoma, and other sarcomas, e.g., soft tissue and bone); tumors of the central and peripheral nervous system (including astrocytoma, neuroblastoma, glioma, glioblastoma, and schwannomas); and other tumors (including melanoma, seminoma, teratocarcinoma, osteosarcoma, xenoderoma pigmentosum, keratoctanthoma, nasopharyngeal cancer, and thyroid follicular cancer). In an embodiment, the cancer overexpressing one or more Bcl-2 family proteins is a p53 wild-type cancer. In an embodiment, the MDM2 inhibitor is the compound of Formula (I) or Formula (II). In an embodiment, the MDM2 inhibitor is selected from the group consisting of a compound of Formula (I), Formula (II), RG7388, Triptolide, HDM201, RG7112, CGM097A, CGM0970B, SJ-172550, SAR405838, MI-773, MX69, YH239-EE, R08994, Nutlin-3, Nutlin-3a, Nutlin-3b, Serdemetan, NSC59984, CHEMBL2386350, MK-8242, DS-3032, DS-3032B, APG-115, MI-1601, and pharmaceutically acceptable salts thereof. In an embodiment, the MDM2 inhibitor is selected from the group consisting of a compound of Formula (I), Formula (II), RG7388, HDM201, RG7112, CGM097A, CGM0970B, SAR405838, MK-8242, DS-3032B, APG-115, MI-1601, and pharmaceutically acceptable salts thereof.

The pharmaceutical compositions are typically formulated to provide a therapeutically effective amount of a MDM2 inhibitor or a pharmaceutically acceptable salt thereof. Where desired, the pharmaceutical compositions contain a pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants. Where desired, other ingredients in addition to a MDM2 inhibitor or a pharmaceutically acceptable salt thereof may be mixed into a preparation or both components may be formulated into separate preparations for use in combination separately or at the same time.

In selected embodiments, the concentration of a MDM2 inhibitor or a pharmaceutically acceptable salt thereof provided in the pharmaceutical compositions of the invention is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v.

In selected embodiments, the concentration of a MDM2 inhibitor or a pharmaceutically acceptable salt thereof provided in the pharmaceutical compositions of the invention is independently greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v.

In selected embodiments, the concentration of a MDM2 inhibitor or a pharmaceutically acceptable salt thereof is independently in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12% or approximately 1% to approximately 10% w/w, w/v or v/v.

In selected embodiments, the concentration of a MDM2 inhibitor or a pharmaceutically acceptable salt thereof is independently in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In selected embodiments, the amount of a MDM2 inhibitor or a pharmaceutically acceptable salt thereof is independently equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g or 0.0001 g.

In selected embodiments, the amount of a MDM2 inhibitor or a pharmaceutically acceptable salt thereof is independently more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g or 10 g.

A MDM2 inhibitor or a pharmaceutically acceptable salt thereof are effective over a wide dosage range. For example, in the treatment of adult humans, dosages independently ranging from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician.

Described below are non-limiting exemplary pharmaceutical compositions and methods for preparing the same.

### Pharmaceutical Compositions for Oral Administration

In selected embodiments, the invention provides a pharmaceutical composition for oral administration comprising a MDM2 inhibitor or a pharmaceutically acceptable salt thereof, and a pharmaceutical excipient suitable for oral administration.

In selected embodiments, the invention provides a solid pharmaceutical composition for oral administration containing: (i) an effective amount of a MDM2 inhibitor or a pharmaceutically acceptable salt thereof, in combination and (ii) a pharmaceutical excipient suitable for oral administration. In selected embodiments, the composition further contains (iii) an effective amount of at least one additional active ingredient.

In selected embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The invention further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

A MDM2 inhibitor or a pharmaceutically acceptable salt thereof can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the essential active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present invention and to minimize precipitation of the compound of the present invention. This can be especially important for compositions for non-oral use, such as for compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, epsilon-caprolactone and isomers thereof, 5-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Examples may include, but are not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In selected embodiments, the invention provides a pharmaceutical composition for injection comprising a MDM2 inhibitor or a pharmaceutically acceptable salt thereof, and a pharmaceutical excipient suitable for injection. Components and amounts of agents in the compositions are as described herein.

The forms in which the compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid and thimerosal.

Sterile injectable solutions are prepared by incorporating a MDM2 inhibitor or a pharmaceutically acceptable salt thereof in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Administration of a MDM2 inhibitor or a pharmaceutically acceptable salt thereof or pharmaceutical composition of these compounds can be effected by any method that enables delivery of the compounds to the site of action. These methods include oral routes, intraduodenal routes, parenteral injection (including intravenous, intra-arterial, subcutaneous, intramuscular, intravascular, intraperitoneal or infusion), topical (e.g., transdermal application), rectal administration, via local delivery by catheter or stent or through inhalation. The combination of compounds can also be administered intraadiposally or intrathecally.

Exemplary parenteral administration forms include solutions or suspensions of active compound in sterile aqueous solutions, for example, aqueous propylene glycol or dextrose solutions. Such dosage forms can be suitably buffered, if desired.

The invention also provides kits. The kits include a MDM2 inhibitor or a pharmaceutically acceptable salt thereof, either alone or in combination in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in selected embodiments, be marketed directly to the consumer. In an embodiment, the invention provides a kit comprising a MDM2 inhibitor or a pharmaceutically acceptable salt thereof for use in the treatment of a cancer overexpressing one or more Bcl-2 family proteins.

### Dosages and Dosing Regimens

The amount of a MDM2 inhibitor or a pharmaceutically acceptable salt thereof administered will be dependent on the human being treated, the severity of the disorder or condition, the rate of administration, the disposition of the compounds and the discretion of the prescribing physician. However, an effective dosage is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day.

In some embodiments, a MDM2 inhibitor or a pharmaceutically acceptable salt thereof is administered in a single dose. Typically, such administration will be by injection—e.g., intravenous injection, in order to introduce the agents quickly. However, other routes may be used as appropriate. A single dose of a MDM2 inhibitor or a pharmaceutically acceptable salt thereof may also be used for treatment of an acute condition.

In some embodiments, a MDM2 inhibitor or a pharmaceutically acceptable salt thereof is administered in multiple doses for treating a cancer overexpressing one or more Bcl-2 family proteins. In an embodiment, a MDM2 inhibitor or a pharmaceutically acceptable salt thereof is administered in multiple doses. In an embodiment, a MDM2 inhibitor or a pharmaceutically acceptable salt thereof is administered in multiple doses by injection—e.g., intravenous injection. In an embodiment, dosing may be once, twice, three times, four times, five times, six times, or more than six times per day. In an embodiment, dosing may be selected from the group consisting of once a day, twice a day, three times a day, four times a day, five times a day, six times a day, once every other day, once weekly, twice weekly, three times weekly, four times weekly, biweekly, and monthly. In other embodiments, a MDM2 inhibitor or a pharmaceutically acceptable salt thereof is administered about once per day to about six times per day. In some embodiments a MDM2 inhibitor or a pharmaceutically acceptable salt thereof is administered once daily, while in other embodiments a MDM2 inhibitor or a pharmaceutically acceptable salt thereof is administered twice daily, and in other embodiments a MDM2 inhibitor or a pharmaceutically acceptable salt thereof is administered three times daily. In some embodiments a MDM2 inhibitor or a pharmaceutically acceptable salt thereof is administered three times a week, including every Monday, Wednesday, and Friday.

Administration of a MDM2 inhibitor or a pharmaceutically acceptable salt thereof may continue as long as necessary. In some embodiments, a MDM2 inhibitor or a pharmaceutically acceptable salt thereof is administered for more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or more days. In some embodiments, a MDM2 inhibitor or a pharmaceutically acceptable salt thereof is administered for less than 28, 14, 7, 6, 5, 4, 3, 2, or 1 day. In some embodiments, a MDM2 inhibitor or a pharmaceutically acceptable salt thereof is administered for about 14 days, about 21 days, about 28 days, about 35 days, about 42 days, about 49 days, or about 56 days. In some embodiments, a MDM2 inhibitor or a pharmaceutically acceptable salt thereof is administered chronically on an ongoing basis—e.g., for the treatment of chronic effects. In another embodiment the administration of a MDM2 inhibitor or a pharmaceutically acceptable salt thereof continues for less than about 7 days. In yet another embodiment the administration continues for more than about 6, 10, 14, 28 days, two months, three months, four months, five months, six months, seven months, eight months, nine months, ten months, eleven months or one year. In some embodiments, the administration continues for more than about one year, two years, three years, four years, or five years. In some embodiments, continuous dosing is achieved and maintained as long as necessary.

In some embodiments, an effective dosage of a MDM2 inhibitor or a pharmaceutically acceptable salt thereof is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg. In some embodiments, an effective dosage of a MDM2 inhibitor or a pharmaceutically acceptable salt thereof is about 15 mg, about 25 mg, about 30 mg, about 50 mg, about 50 mg, about 75 mg, about 90 mg, about 100 mg, about 120 mg, about 125 mg, about 150 mg, about 175 mg, about 180 mg, about 200 mg, about 225 mg, about 240 mg, about 250 mg, about 275 mg, about 300 mg, about 325 mg, about 350 mg, about 360 mg, about 375 mg, about 400 mg, about 425 mg, about 450 mg, about 475 mg, about 480 mg, or about 500 mg. In some embodiments, an effective dosage of a MDM2 inhibitor or a pharmaceutically acceptable salt thereof is 15 mg, 25 mg, 30 mg, 50 mg, 60 mg, 75 mg, 90 mg, 100 mg, 120 mg, 150 mg, 175 mg, 180 mg, 200 mg, 225 mg, 240 mg, 250 mg, 275 mg, 300 mg, 325 mg, 350 mg, 360 mg, 375 mg, and 480 mg.

In some embodiments, an effective dosage of a MDM2 inhibitor or a pharmaceutically acceptable salt thereof is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg. In some embodiments, an effective dosage of a MDM2 inhibitor or a pharmaceutically acceptable salt thereof is about 0.35 mg/kg, about 0.7 mg/kg, about 1 mg/kg, about 1.4 mg/kg, about 1.8 mg/kg, about 2.1 mg/kg, about 2.5 mg/kg, about 2.85 mg/kg, about 3.2 mg/kg, or about 3.6 mg/kg.

In some embodiments, a MDM2 inhibitor or a pharmaceutically acceptable salt thereof is administered at a dosage of 10 to 500 mg BID, including a dosage of 15 mg, 25 mg, 30 mg, 50 mg, 60 mg, 75 mg, 90 mg, 100 mg, 120 mg, 150 mg, 175 mg, 180 mg, 200 mg, 225 mg, 240 mg, 250 mg, 275 mg, 300 mg, 325 mg, 350 mg, 360 mg, 375 mg, and 480 mg BID.

In some embodiments, a MDM2 inhibitor or a pharmaceutically acceptable salt thereof is administered at a dosage of 10 to 500 mg QD, including a dosage of 15 mg, 25 mg, 30 mg, 50 mg, 60 mg, 75 mg, 90 mg, 100 mg, 120 mg, 150 mg, 175 mg, 180 mg, 200 mg, 225 mg, 240 mg, 250 mg, 275 mg, 300 mg, 325 mg, 350 mg, 360 mg, 375 mg, and 480 mg QD.

An effective amount of a MDM2 inhibitor or a pharmaceutically acceptable salt thereof may be administered in either single or multiple doses by any of the accepted modes of administration of agents having similar utilities, including buccal, sublingual, and transdermal routes, by intra-arterial injection, intravenously, parenterally, intramuscularly, subcutaneously or orally.

In some embodiments, a MDM2 inhibitor or a pharmaceutically acceptable salt thereof is administered to a human subject intermittently, known as intermittent administration. By “intermittent administration”, it is meant a period of administration of a therapeutically effective dose of a MDM2 inhibitor or a pharmaceutically acceptable salt thereof, followed by a time period of discontinuance, which is then followed by another administration period and so on. In each administration period, the dosing frequency can be independently select from three times daily, twice daily, daily, once weekly, twice weekly, three times weekly, four times weekly, five times weekly, six times weekly or monthly. In an embodiment, the MDM2 inhibitor is the compound of Formula (I) or Formula (II). In an embodiment, the MDM2 inhibitor is selected from the group consisting of a compound of Formula (I), Formula (II), RG7388, Triptolide, HDM201, RG7112, CGM097A, CGM0970B, SJ-172550, SAR405838, MI-773, MX69, YH239-EE, R08994, Nutlin-3, Nutlin-3a, Nutlin-3b, Serdemetan, NSC59984, CHEMBL2386350, MK-8242, DS-3032, DS-3032B, APG-115, MI-1601, and pharmaceutically acceptable salts thereof. In an embodiment, the MDM2 inhibitor is selected from the group consisting of a compound of Formula (I), Formula (II), RG7388, HDM201, RG7112, CGM097A, CGM0970B, SAR405838, MK-8242, DS-3032B, APG-115, MI-1601, and pharmaceutically acceptable salts thereof.

By “period of discontinuance” or “discontinuance period” or “rest period”, it is meant to the length of time when discontinuing of the administration of a MDM2 inhibitor or a pharmaceutically acceptable salt thereof. The time period of discontinuance may be longer or shorter than the administration period or the same as the administration period. During the discontinuance period, other therapeutic agents other than a MDM2 inhibitor or a pharmaceutically acceptable salt thereof may be administered.

In an embodiment, a MDM2 inhibitor or a pharmaceutically acceptable salt thereof is administered to a human subject in need thereof for treating a cancer overexpressing one or more Bcl-2 family proteins for a first administration period, then followed by a discontinuance period, then followed by a second administration period, and so on. The first administration period, the second administration period, and the discontinuance period are independently selected from the group consisting of more than 1, 2, 3, 4, 5, 6, 7, 8, 9, 10, 11, 12, 13, 14, 15, 16, 17, 18, 19, 20, 21, 22, 23, 24, 25, 26, 27, 28, 29, one month, five weeks, six weeks, seven weeks, two months, nine weeks, ten weeks, elven weeks, three months, thirteen weeks, fourteen weeks, fifteen weeks, four months, and more days, in which a MDM2 inhibitor or a pharmaceutically acceptable salt thereof is administered to a human subject three times daily, twice daily, daily, once weekly, twice weekly, three times weekly, four times weekly, five times weekly, six times weekly or monthly. In an embodiment, the first administration period is at same length as the second administration period. In an embodiment, the first administration period is shorter than the second administration period. In an embodiment, the first administration period is longer than the second administration period. In an embodiment, the first administration period and the second administration period are about one week, in which a MDM2 inhibitor or a pharmaceutically acceptable salt thereof is administered to a human subject daily; and the discontinuance period is about two weeks. In an embodiment, the first administration period and the second administration period are about three weeks, in which a MDM2 inhibitor or a pharmaceutically acceptable salt thereof is administered to a human subject daily; and the discontinuance period is about two weeks. In an embodiment, the first administration period and the second administration period are about three weeks, in which a MDM2 inhibitor or a pharmaceutically acceptable salt thereof is administered to a human subject weekly; and the discontinuance period is about two weeks. In an embodiment, the first administration period and the second administration period are about four weeks, in which a MDM2 inhibitor or a pharmaceutically acceptable salt thereof is administered to a human subject daily; and the discontinuance period is about two weeks. In an embodiment, the first administration period and the second administration period are about four weeks, in which a MDM2 inhibitor or a pharmaceutically acceptable salt thereof is administered to a human subject weekly; and the discontinuance period is about two weeks. In an embodiment, the MDM2 inhibitor is the compound of Formula (I) or Formula (II). In an embodiment, the MDM2 inhibitor is selected from the group consisting of a compound of Formula (I), Formula (II), RG7388, Triptolide, HDM201, RG7112, CGM097A, CGM0970B, SJ-172550, SAR405838, MI-773, MX69, YH239-EE, R08994, Nutlin-3, Nutlin-3a, Nutlin-3b, Serdemetan, NSC59984, CHEMBL2386350, MK-8242, DS-3032, DS-3032B, APG-115, MI-1601, and pharmaceutically acceptable salts thereof. In an embodiment, the MDM2 inhibitor is selected from the group consisting of a compound of Formula (I), Formula (II), RG7388, HDM201, RG7112, CGM097A, CGM0970B, SAR405838, MK-8242, DS-3032B, APG-115, MI-1601, and pharmaceutically acceptable salts thereof. In an embodiment, the cancer overexpressing one or more Bcl-2 family proteins is a p53 wild-type cancer.

In an embodiment, a composition comprising a MDM2 inhibitor or a pharmaceutically acceptable salt thereof is administered to a human subject in need thereof for treating a cancer overexpressing one or more Bcl-2 family proteins on days 1-7 of a 21-day cycle (on days 8-21, the MDM2 inhibitor is not administered) for a period selected from 3 weeks, 6 weeks, 9 weeks, 12 weeks, 15 weeks, 18 weeks, 21 weeks, 24 weeks, 27 weeks, 30 weeks, 33 weeks, 36 weeks, 39 weeks, 42 weeks, 45 weeks, 48 weeks, 51 weeks, 54 weeks, 57 weeks, 60 weeks, 63 weeks, 66 weeks, 69 weeks, 72 weeks, 75 weeks, 78 weeks, 81 weeks, 84 weeks, 87 weeks, 90 weeks, 93 weeks, 96 weeks, 99 weeks, 102 weeks, 105 weeks, 108 weeks, 111 weeks, 114 weeks, 117 weeks, 120 weeks, 123 weeks, 126 weeks, 129 weeks, 132 weeks, 135 weeks, 138 weeks, 141 weeks, 144 weeks, 147 weeks, 150 weeks, 153 weeks, and 156 weeks, wherein the MDM2 inhibitor is selected from the group consisting of a compound of Formula (I), Formula (II), RG7388, Triptolide, HDM201, RG7112, CGM097A, CGM0970B, SJ-172550, SAR405838, MI-773, MX69, YH239-EE, R08994, Nutlin-3, Nutlin-3a, Nutlin-3b, Serdemetan, NSC59984, CHEMBL2386350, MK-8242, DS-3032, DS-3032B, APG-115, MI-1601, and pharmaceutically acceptable salts thereof, wherein the cancer overexpressing one or more Bcl-2 family proteins is selected from the group consisting of carcinomas such as cancer of the bladder, breast, colon, rectum, kidney, liver, lung (small cell lung cancer, and non-small-cell lung cancer), esophagus, gall-bladder, ovary, pancreas, stomach, cervix, thyroid, prostate, head and neck, and skin (including basal and squamous cell carcinoma, melanoma skin cancer, Merkel cell carcinoma, Kaposi Sarcoma, skin lymphomas); hematopoietic tumors of lymphoid lineage (including leukemia, acute lymphocytic leukemia, chronic myelogenous leukemia, acute lymphoblastic leukemia, B-cell lymphoma, T-cell-lymphoma, Hodgkin's lymphoma, non-Hodgkin's lymphoma, hairy cell lymphoma and Burkett's lymphoma); hematopoietic tumors of myeloid lineage (including acute and chronic myelogenous leukemias, myelodysplastic syndrome and promyelocytic leukemia); tumors of mesenchymal origin (including fibrosarcoma and rhabdomyosarcoma, and other sarcomas, e.g., soft tissue and bone); tumors of the central and peripheral nervous system (including astrocytoma, neuroblastoma, glioma, glioblastoma, and schwannomas); and other tumors (including melanoma, seminoma, teratocarcinoma, osteosarcoma, xenoderoma pigmentosum, keratoctanthoma, nasopharyngeal cancer, and thyroid follicular cancer). In an embodiment, the MDM2 inhibitor is selected from the group consisting of a compound of Formula (I), Formula (II), RG7388, HDM201, RG7112, CGM097A, CGM0970B, SAR405838, MK-8242, DS-3032B, APG-115, MI-1601, and pharmaceutically acceptable salts thereof. In an embodiment, the cancer overexpressing one or more Bcl-2 family proteins is a p53 wild-type cancer.

In an embodiment, a MDM2 inhibitor or a pharmaceutically acceptable salt thereof for use in the treatment of a cancer overexpressing one or more Bcl-2 family proteins, wherein the MDM2 inhibitor or a pharmaceutically acceptable salt thereof is administered to a human subject in need thereof on days 1-7 of a 28-day cycle (on days 8-28, the MDM2 inhibitor is not administered) for a period selected from 4 weeks, 8 weeks, 12 weeks, 16 weeks, 20 weeks, 24 weeks, 28 weeks, 32 weeks, 36 weeks, 40 weeks, 44 weeks, 48 weeks, 52 weeks, 56 weeks, 60 weeks, 64 weeks, 68 weeks, 72 weeks, 76 weeks, 80 weeks, 84 weeks, 88 weeks, 92 weeks, 96 weeks, 100 weeks, 104 weeks, 108 weeks, 112 weeks, 116 weeks, 120 weeks, 124 weeks, 128 weeks, 132 weeks, 136 weeks, 140 weeks, 144 weeks, 148 weeks, 152 weeks, and 156 weeks, wherein the MDM2 inhibitor is selected from the group consisting of a compound of Formula (I), Formula (II), RG7388, Triptolide, HDM201, RG7112, CGM097A, CGM0970B, SJ-172550, SAR405838, MI-773, MX69, YH239-EE, R08994, Nutlin-3, Nutlin-3a, Nutlin-3b, Serdemetan, NSC59984, CHEMBL2386350, MK-8242, DS-3032, DS-3032B, APG-115, MI-1601, and pharmaceutically acceptable salts thereof, wherein the cancer overexpressing one or more Bcl-2 family proteins is selected from the group consisting of carcinomas such as cancer of the bladder, breast, colon, rectum, kidney, liver, lung (small cell lung cancer, and non-small-cell lung cancer), esophagus, gall-bladder, ovary, pancreas, stomach, cervix, thyroid, prostate, head and neck, and skin (including basal and squamous cell carcinoma, melanoma skin cancer, Merkel cell carcinoma, Kaposi Sarcoma, skin lymphomas); hematopoietic tumors of lymphoid lineage (including leukemia, acute lymphocytic leukemia, chronic myelogenous leukemia, acute lymphoblastic leukemia, B-cell lymphoma, T-cell-lymphoma, Hodgkin's lymphoma, non-Hodgkin's lymphoma, hairy cell lymphoma and Burkett's lymphoma); hematopoietic tumors of myeloid lineage (including acute and chronic myelogenous leukemias, myelodysplastic syndrome and promyelocytic leukemia); tumors of mesenchymal origin (including fibrosarcoma and rhabdomyosarcoma, and other sarcomas, e.g., soft tissue and bone); tumors of the central and peripheral nervous system (including astrocytoma, neuroblastoma, glioma, glioblastoma, and schwannomas); and other tumors (including melanoma, seminoma, teratocarcinoma, osteosarcoma, xenoderoma pigmentosum, keratoctanthoma, nasopharyngeal cancer, and thyroid follicular cancer). In an embodiment, the MDM2 inhibitor is selected from the group consisting of a compound of Formula (I), Formula (II), RG7388, HDM201, RG7112, CGM097A, CGM0970B, SAR405838, MK-8242, DS-3032B, APG-115, MI-1601, and pharmaceutically acceptable salts thereof. In an embodiment, the MDM2 inhibitor is a compound of Formula (I) or Formula (II) and a pharmaceutically acceptable salt thereof; and the MDM 2 inhibitor is orally administered at a dose of 120 mg or 240 mg once a day (QD). In an embodiment, the cancer overexpressing one or more Bcl-2 family proteins is a p53 wild-type cancer.

In an embodiment, a composition comprising a MDM2 inhibitor or a pharmaceutically acceptable salt thereof for use in the treatment of a cancer overexpressing one or more Bcl-2 family proteins, wherein the composition is administered to a human subject in need thereof on days 1-7 of a 28-day cycle (on days 8-28, the MDM2 inhibitor is not administered) for a period selected from 4 weeks, 8 weeks, 12 weeks, 16 weeks, 20 weeks, 24 weeks, 28 weeks, 32 weeks, 36 weeks, 40 weeks, 44 weeks, 48 weeks, 52 weeks, 56 weeks, 60 weeks, 64 weeks, 68 weeks, 72 weeks, 76 weeks, 80 weeks, 84 weeks, 88 weeks, 92 weeks, 96 weeks, 100 weeks, 104 weeks, 108 weeks, 112 weeks, 116 weeks, 120 weeks, 124 weeks, 128 weeks, 132 weeks, 136 weeks, 140 weeks, 144 weeks, 148 weeks, 152 weeks, and 156 weeks, wherein the MDM2 inhibitor is selected from the group consisting of a compound of Formula (I), Formula (II), RG7388, Triptolide, HDM201, RG7112, CGM097A, CGM0970B, SJ-172550, SAR405838, MI-773, MX69, YH239-EE, R08994, Nutlin-3, Nutlin-3a, Nutlin-3b, Serdemetan, NSC59984, CHEMBL2386350, MK-8242, DS-3032, DS-3032B, APG-115, MI-1601, and pharmaceutically acceptable salts thereof. In an embodiment, the MDM2 inhibitor is selected from the group consisting of a compound of Formula (I), Formula (II), RG7388, HDM201, RG7112, CGM097A, CGM0970B, SAR405838, MK-8242, DS-3032B, APG-115, MI-1601, and pharmaceutically acceptable salts thereof. In an embodiment, the MDM2 inhibitor is a compound of Formula (I) or Formula (II) and a pharmaceutically acceptable salt thereof; and the MDM 2 inhibitor is orally administered at a dose of 120 mg or 240 mg once a day (QD). In an embodiment, the cancer overexpressing one or more Bcl-2 family proteins is a p53 wild-type cancer.

### Selection of Patients Overexpressing One or More Bcl-2 Family Proteins

Embodiments of the invention include diagnostic tests, testing methods and assays to determine the presence of a cancer overexpressing one or more Bcl-2 family proteins selected from the group consisting of Bcl-2, Bcl-XL, Bcl-W, Mcl-1, Bfl-1/A1, and Bcl-B. In some embodiments, the invention includes testing methods and assays to determine the presence of a cancer overexpressing one or more Bcl-2 family proteins selected from the group consisting of Bcl-2, Bcl-XL, Bcl-W, Mcl-1, and Bfl-1/A1. In some embodiments, patients with high expression of one or more Bcl-2 family proteins including but not limited to Bcl-xL, Bcl-2, Bcl-w, and BAD relative to a normal control can be selected for MDM2 inhibitor therapy. Further, patients with low expression a particular Bcl-2 family proteins can be excluded from MDM2 inhibitor therapy.

Therefore, in some embodiments, the cancer overexpressing one or more Bcl-2 family proteins described herein can be characterized by overexpression of Bcl-2 or Bcl-XL or BAD or Bcl-w.

In some embodiments, the cancer overexpressing one or more Bcl-2 family proteins is characterized by overexpression of Bcl-2, but not BAD. In some embodiments, the cancer overexpressing one or more Bcl-2 family proteins is characterized by overexpression of Bcl-2, but not Bcl-XL. In some embodiments, the cancer overexpressing one or more Bcl-2 family proteins is characterized by overexpression of Bcl-2, but not Bcl-w. In some embodiments, the cancer overexpressing one or more Bcl-2 family proteins is characterized by overexpression of Bcl-2, but not BAD, Bcl-XL and Bcl-w.

In some embodiments, the cancer overexpressing one or more Bcl-2 family proteins is characterized by overexpression one or more Bcl-2 family proteins selected from the group consisting of Bcl-XL, Bcl-2, and Bcl-w.

Various types of assays and tests may be used, including, but not limited to immunoassays, spectrometry, mass spectrometry, Matrix Assisted Laser Desorption/Ionization Time-of-Flight (MALDI-TOF) Mass Spectrometry, microscopy, northern blot, western blot, southern blot, isoelectric focusing, SDS-PAGE, PCR, RT-PCR, gel electrophoresis, protein microarray, DNA microarray, and antibody microarray, or combinations thereof. The level of Bcl-2 gene expression is determined using an immunoassay. The immunoassay may be an enzyme linked immunoassay (ELISA), a sandwich assay, a radioimmunoassay, a Western Blot, an immunoprecipitation assay, or an agglutination assay.

The presence, and possibly even the level, of a particular Bcl-2 protein in a sample may also be determined by using mass spectrometry techniques. Mass spectrometry techniques may be used to detect gas phase ions which correlate to specific proteins or parts of proteins, such as, trypsin peptides. Examples of mass spectrometers include time of flight, magnetic sector, quadruple filter, ion trap, ion cyclotron resonance, electrostatic sector analyser and hybrids of these. The mass spectrometer may use laser desorption/ionization.

MALDI (Matrix-assisted laser desorption/ionization) peptide mass fingerprinting may be used to identify the presence of particular Bcl-2 protein from 2D gel analysis and RPE/SDS PAGE analysis. MALDI-MS and ANN analysis may be used to profile identified proteins and tryptic biomarker signatures to determine that certain malignant myeloid cells are susceptible to MDM2 inhibition.

In a particular embodiment, disclosed is the use of immobilized antibodies which target and identify one or more Bcl-2 family proteins, or a combination thereof. These could be in the form of an ELISA test, in a non-limiting embodiment, which may quantify the relative expression levels. High expression of one or more Bcl-2 family proteins would indicate susceptibility to MDM2 inhibitor therapy. Low expression would indicate decreased sensitivity to MDM2 inhibitor therapy. Endpoints could be measured in terms of measurement by immunohistochemistry (IHC) or by Western blot analysis using a standard protein like beta actin for normalization and quantification.

The ability to easily quantify Bcl-2 gene expression would provide guidance for treatment using effective and specific MDM2 inhibitor treatment therapies. MDM2 inhibitor therapy will be more effective against malignant cells with high expression of Bcl-2 gene. A multitude of MDM2 inhibitor therapies can be enhanced by the pre-selection of patients who will best respond to the therapies identified with the tests and methods described herein.

### EXAMPLES

The following examples are offered to illustrate, but not to limit the invention.

### Example 1

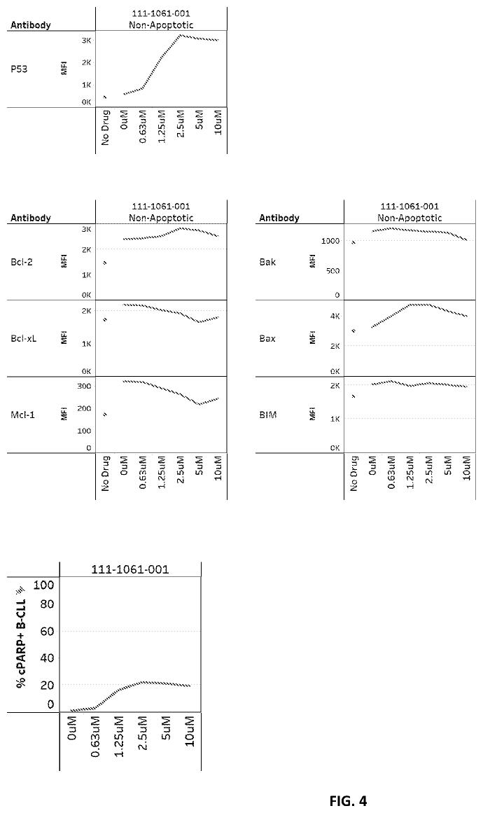

The effect of the compound of Formula (I) on the expression of p53 along with anti-apoptotic and pro-apoptotic proteins was assessed in tumors cells isolated from patients diagnosed with CLL. Blood samples were collected prior to the patient receiving any treatment. CLL tumor cells were isolated and incubated on HS-5 stromal cells for 24 hours and then treated with the compound of Formula (I) for 24 hours. p53 and Bcl-2 family proteins were measured by intracellular flow cytometry and shown as median fluorescent intensity (MFI). The percent cPARP+ is shown above the level of basal spontaneous apoptosis. CLL tumor cells were cultured on HS-5 stromal cells were then treated with an increasing concentrations of the compound of Formula (I) for 24 hours. The result was an increase in intracellular P53 levels along with a decrease in anti-apoptotic Bcl-XL and Mcl-1 levels, and an increase in pro-apoptotic Bax. This led to increased cell apoptosis in a dose-dependent manner (FIG. 4). Hence, the compound of Formula (I) led to increased expression of p53, along with pro-apoptotic proteins Bax and Bak, and decreased expression of anti-apoptotic proteins Bcl-2, Bcl-xL and Mcl-1, which promoted apoptosis and subsequent tumor cell death.

### Example 2

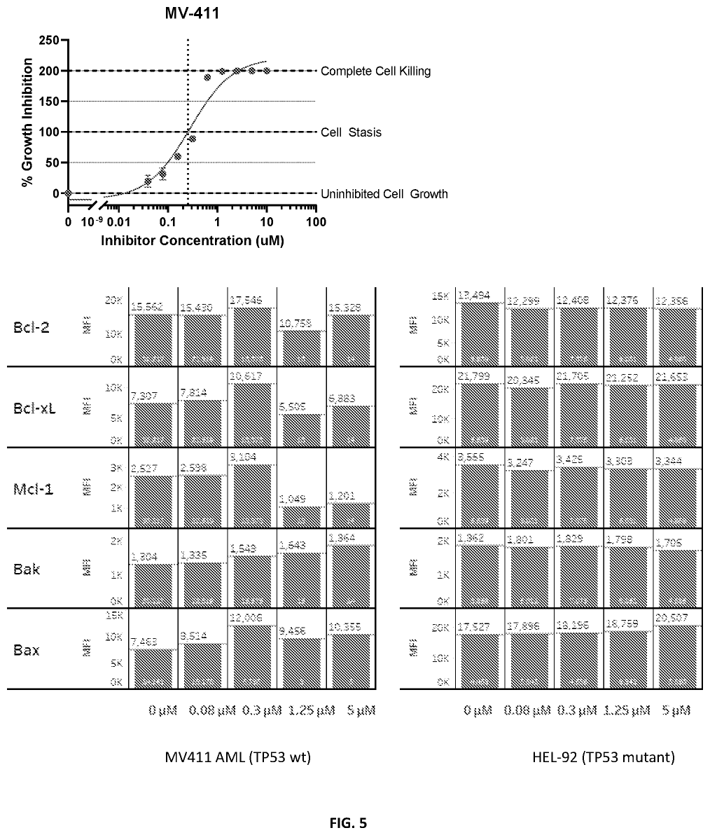

The effect of the compound of Formula (I) on inducing apoptosis along with the expression of anti-apoptotic and pro-apoptotic protein expression in the MV-411 AML cell was assessed. The MV-411 AML cell line (TP53 wild-type) was cultured for 72 hours with increasing concentrations of compound of Formula (I) and cell proliferation was measured using a Cell TiterGlo cell proliferation assay (FIG. 5(A)), described in Canon et al., The MDM2 Inhibitor AMG 232 Demonstrates Robust Antitumor Efficacy and Potentiates the Activity of p53-Inducing Cytotoxic AgentsAMG 232 Regresses Tumors and Enhances Chemotherapy Efficacy. Molecular Cancer Therapeutics. 2015 Mar. 1; 14(3):649-58. After 72 hours of culture with the compound of Formula (I), MV-411 (TP53 wt) and HEL-92 cells (TP53 mutant) were stained with antibodies against various Bcl-2 family proteins and data acquired using intracellular flow cytometry (FIG. 5(B)). In MV-411 cells, treatment with the compound of Formula (I) resulted in a decrease in anti-apoptotic Bcl-2 family proteins and an increase in the pro-apoptotic effector proteins (Bak and Bax) once cells were treated above the concentration to induce cell stasis. In HEL-92 cells, no changes in Bcl-2 family proteins were observed due to a TP53 mutation rendering the p53 protein inactive.

### Example 3

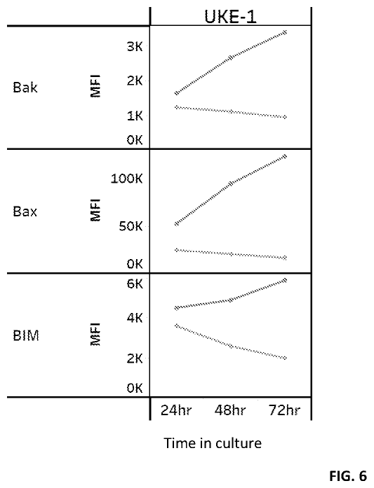

The compound of Formula (I) induces Bak, Bax and BIM expression in a myelofibrosis cell line. The UKE-1 myelofibrosis cell line (TP53 wild-type) was cultured for 24, 48 and 72 hours with 2.5 pM of compound (equivalent to levels observed in vivo following administration in patients) and stained with antibodies against various pro-apoptotic protein using intracellular flow cytometry techniques. The compound of Formula (I) induced protein expression of Bak, Bax, and BIM in a time-dependent manner (FIG. 6). The upper line is cells treated with the compound of Formula (I) while the lower line is a control (no treatment).

### Example 4

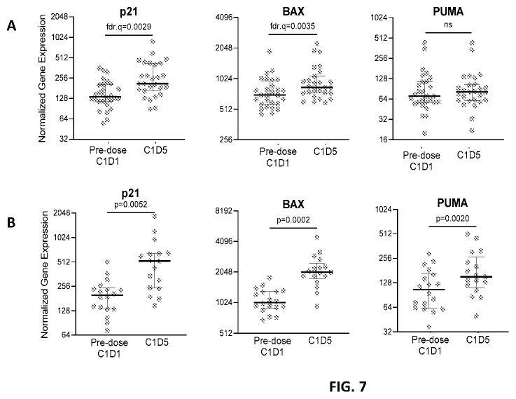

Treatment of patients with the compound of Formula (I) induces p21, Bax and Puma expression in vivo in patients with R/R myelofibrosis or R/R AML. mRNA expression in cells isolated from patients was evaluated pre-dosing on day 1 and 24 hours after the first course of treatment with the compound of Formula (I) in patients with R/R myelofibrosis (FIG. 7(A)). The compound of Formula (I) is administered at 120 mg for 7 days for Cohort 1, 240 mg for 7 days for Cohort 2, 240 mg for 7 days for Cohort 3, and 240 mg for 5 days for Cohort 4. Samples taken at pre-dose on Cycle 1, Day 1 (C1D1) and Cycle 1, Day 5 (C1D5). FIG. 7(A) demonstrates increased expression of p21 and Bax in patients with R/R myelofibrosis following treatment with the compound of Formula (I).

For patients with R/R AML, the compound of Formula (I) was administered at 120 mg (n=3), 180 mg (n=2), 240 mg (n=8), or 360 mg (n=4) daily for QD for seven days in dose-escalation phase, with different ‘off-treatment’ duration depending on cohort assignments. Samples taken at pre-dose on Cycle 1, Day 1 (C1D1) and Cycle 1, Day 5 (C1D5). FIG. 7(B) demonstrates increased expression of p21, Bax and Puma in patients with R/R AML following treatment with the compound of Formula (I) (abbreviations: fdr.q, false discovery rate adjusted significance value for change in normalized gene expression).

### Example 5

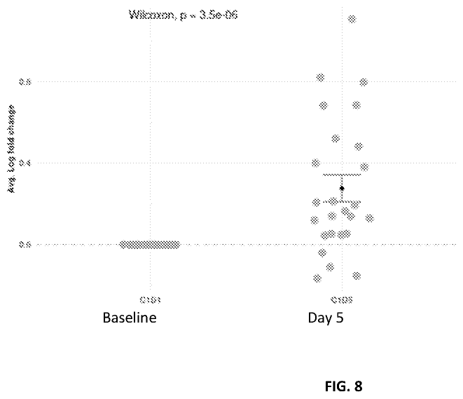

Treatment of patients with R/R AML with the compound of Formula (I) induces expression of genes involved in the Bcl-2 apoptotic pathway. In clinical studies testing the effect of the compound of Formula (I) in R/R AML patients, treatment induced the expression of genes involved in the BCL2 family/apoptosis pathway in cells isolated from peripheral blood. FIG. 8 illustrates induced expression of Bcl-2 genes in PBMC following treatment of R/R AML patients with the compound of Formula (I). Eighteen genes were included in analysis: BAD, BAX, BCL2L1, MCL1, BCL2, BID, CAPN2, CASP10, CASP3, CASP7, CASP8, CASP9, TNFRSF10A, TNFRSF10B, TP53, PUMA, NOXA and STAT3. Experimental data was obtained similar to the method set forth in Example 4.

While exemplary embodiments have been shown and described above, it will be apparent to those skilled in the art that modifications and variations could be made without departing from the scope of the present disclosure as defined by the appended claims.

1. A method of treating a cancer overexpressing one or more Bcl-2 anti-apoptotic/pro-survival family proteins in a human subject with a wild-type p53 gene comprising the step of administering to a human subject in need thereof a therapeutically effective amount of a MDM2 inhibitor on days 1 through 7 followed by no administration of the MDM2 inhibitor on days 8 through 21, wherein the MDM2 inhibitor is a compound of Formula (I) or Formula (II): 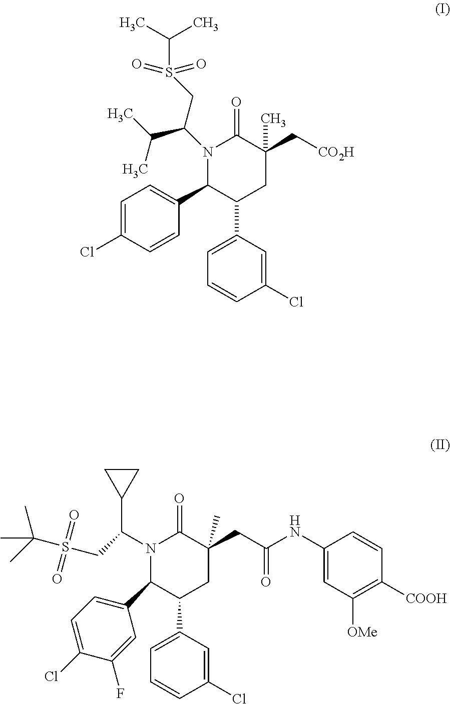 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1 ; COc1cc(NC(=O)C[C@@]2(C)C[C@H](c3cccc(Cl)c3)[C@@H](c3ccc(Cl)c(F)c3)N([C@H](CS(=O)(=O)C(C)(C)C)C3CC3)C2=O)ccc1C(=O)O]
or a pharmaceutically acceptable salt thereof, wherein the one or more Bcl-2 anti-apoptotic/pro-survival family proteins are selected from the group consisting of Bcl-2, Bcl-XL, Bcl-W, Mcl-1, Bfl-1/A1, and Bcl-B.

2. The method of claim 1, wherein the MDM2 inhibitor is administered again after day 21 for 7 days followed by no administration of the MDM2 inhibitor for 14 days.

3. The method of claim 1, wherein the cancer is selected from the group consisting of Merkel cell carcinoma, diffuse large B-cell lymphoma (DLBCL), mantle cell lymphoma (MCL), metastatic prostate cancer, small cell lung cancer (SCLC), Burkett's lymphoma, T-cell lymphoma, acute myeloid leukemia (AML) and myelofibrosis.

4. The method of claim 3, wherein the T-cell lymphoma is peripheral T-cell lymphoma or aggressive T cell lymphoma.

5. The method of claim 3, wherein the cancer is a relapsed/refractory cancer.

6. The method of claim 3, wherein the small cell lung cancer is a p53 wild-type lung cancer.

7. The method of claim 1, wherein the cancer is a B cell hematological malignancy.

8. The method of claim 7, wherein the B cell hematological malignancy is selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, and Waldenström's macroglobulinemia (WM).

9. The method of any one of claims 1 to 8, wherein the human subject is previously treated with immunotherapy.

10. The method of claim 9, wherein the immunotherapy is an ex vivo cell therapy selected from the group consisting of tumor-infiltrating lymphocytes (TILs), T-cell receptor (TCR)-engineered peripheral blood lymphocytes (PBL) and chimeric antigen receptor ((CAR)-engineered PBL).

11. The method of claim 9, wherein the immunotherapy is an immune checkpoint protein inhibitor therapy.

12. The method of claim 11, wherein the immune checkpoint protein inhibitor is an anti-PD-L1 antibody selected from the group consisting of BMS-936559, durvalumab, atezolizumab, avelumab, MPDL3280A, MEDI4736, MSB0010718C, MDX1105-01, and fragments, conjugates, biosimilars, or variants thereof.

13. The method of claim 11, wherein the immune checkpoint protein inhibitor is an anti-PD-1 antibody selected from group consisting of nivolumab, tslelizimab, dostarlimab, pembrolizumab, pidilizumab, cemiplimab-rwlc, AMP-224, AMP-514, PDR001, and fragments, conjugates, biosimilars, or variants thereof.

14. The method of claim 11, wherein the immune checkpoint protein inhibitor is an anti-CTLA-4 antibody selected from the group consisting of ipilimumab, tremelimumab, and fragments, conjugates, biosimilars, or variants thereof.

15. The method of claim 9, wherein the immunotherapy is a T-cell engager selected from catumaxomab, FBTA05, Ertumaxomab, Ektomun, blinatumomab, solitomab, and fragments, conjugates, biosimilars, or variants thereof.

16. The method of any one of claims 1 to 15, compound of Formula (I) or Formula (II) is in a crystalline form.

17. The method of any one of claims 1 to 15, wherein the compound of Formula (I) or Formula (II) is in a free form.

18. The method of any one of claims 1 to 15, wherein the MDM2 inhibitor is a pharmaceutically acceptable salt of a compound of Formula (I) or Formula (II).

19. The method of any one of claims 1 to 15, wherein the compound of Formula (I) or Formula (II) is in an amorphous form.

20. The method of any one of claims 1 to 19, wherein the compound of Formula (I) or Formula (II) is administered once daily at a dose selected from the group consisting of 15 mg, 25 mg, 30 mg, 50 mg, 60 mg, 75 mg, 90 mg, 100 mg, 120 mg, 150 mg, 175 mg, 180 mg, 200 mg, 225 mg, 240 mg, 250 mg, 275 mg, 300 mg, 325 mg, 350 mg, 360 mg, 375 mg, 420 mg, and 480 mg.

21. The method of any one of claims 1 to 19, wherein the compound of Formula (I) or Formula (II) is administered twice daily at a dose selected from the group consisting of 15 mg, 25 mg, 30 mg, 50 mg, 60 mg, 75 mg, 90 mg, 100 mg, 120 mg, 150 mg, 175 mg, 180 mg, 200 mg, 225 mg, 240 mg, 250 mg, 275 mg, 300 mg, 325 mg, 350 mg, 360 mg, 375 mg, 420 mg, and 480 mg.

22. The method of any one of claims 1 to 21, wherein the compound of Formula (I) or Formula (II) is orally administered.

23. The method of any one of claims 1 to 22, wherein the therapeutically effective amount of the MDM2 inhibitor is 120 mg.

24. The method of any one of claims 1-23, wherein the human subject has a Ki67 protein proliferation rate more than 10%.

25. The method of any one of claims 1-23, wherein the human subject has a Ki67 protein proliferation rate more than 15%.

26. The method of any one of claims 1-23, wherein the human subject has a Ki67 protein proliferation rate more than 20%.

27. The method of any one of claims 1-23, wherein the human subject has a Ki67 protein proliferation rate more than 25%.

28. The method of any one of claims 1-27, the cancer is characterized by overexpression of Bcl-2.

29. The method of any one of claims 1-27, the cancer is characterized by overexpression of Bcl-XL.

30. The method of any one of claims 1-27, the cancer is characterized by overexpression of Mcl-1.

31. The method of any one of claims 1-27, the cancer is characterized by overexpression of Bcl-w.

32. A MDM2 inhibitor for use in treating a cancer overexpressing one or more Bcl-2 anti-apoptotic/pro-survival family proteins with a wild-type p53 gene, wherein the MDM2 inhibitor is a compound of Formula (I) or Formula (II): 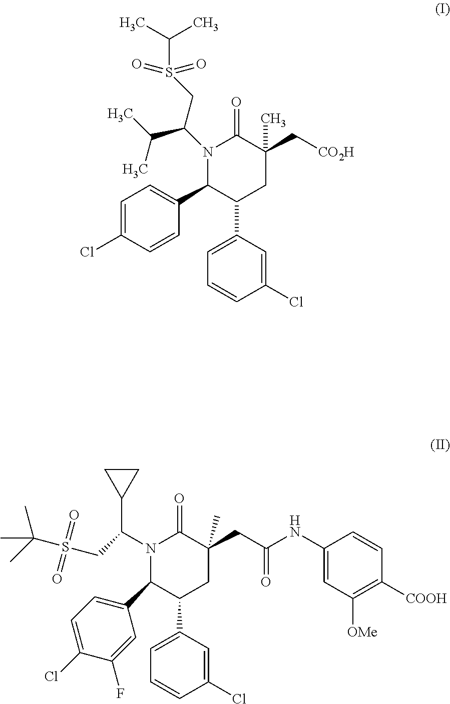 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1 ; COc1cc(NC(=O)C[C@@]2(C)C[C@H](c3cccc(Cl)c3)[C@@H](c3ccc(Cl)c(F)c3)N([C@H](CS(=O)(=O)C(C)(C)C)C3CC3)C2=O)ccc1C(=O)O]
or a pharmaceutically acceptable salt thereof, wherein the one or more Bcl-2 anti-apoptotic/pro-survival family proteins are selected from the group consisting of Bcl-2, Bcl-XL, Bcl-W, Mcl-1, Bfl-1/A1, and Bcl-B, wherein the MDM2 inhibitor is administered on days 1 through 7 followed by no administration of the MDM2 inhibitor on days 8 through 21.

33. The use of claim 32, wherein the cancer is selected from the group consisting of Merkel cell carcinoma, diffuse large B-cell lymphoma (DLBCL), mantle cell lymphoma (MCL), metastatic prostate cancer, small cell lung cancer (SCLC), Burkett's lymphoma, T-cell lymphoma, acute myelogenous leukemia (AML) and myelofibrosis (MF).

34. The use of claim 33, wherein the T-cell lymphoma is peripheral T-cell lymphoma or aggressive T-cell lymphoma.

35. The use of claim 33, wherein the cancer is a relapsed/refractory cancer.

36. The use of claim 33, wherein the small cell lung cancer is a p53 wild-type lung cancer.

37. The use of claim 32, wherein the cancer is a B cell hematological malignancy.

38. The use of claim 37, wherein the B cell hematological malignancy is selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, and Waldenström's macroglobulinemia (WM).

39. The use of any one of claims 32 to 38, wherein the human subject is previously treated with immunotherapy.

40. The use of any one of claims 32 to 39, compound of Formula (I) or Formula (II) is in a crystalline form.

41. The use of any one of claims 32 to 39, wherein the compound of Formula (I) or Formula (II) is in a free form.

42. The use of any one of claims 32 to 39, wherein the MDM2 inhibitor is a pharmaceutically acceptable salt of a compound of Formula (I) or Formula (II).

43. The use of any one of claims 32 to 39, wherein the compound of Formula (I) or Formula (II) is in an amorphous form.

44. The use of any one of claims 32 to 43, wherein the compound of Formula (I) or Formula (II) is administered once daily at a dose selected from the group consisting of 15 mg, 25 mg, 30 mg, 50 mg, 60 mg, 75 mg, 90 mg, 100 mg, 120 mg, 150 mg, 175 mg, 180 mg, 200 mg, 225 mg, 240 mg, 250 mg, 275 mg, 300 mg, 325 mg, 350 mg, 360 mg, 375 mg, and 480 mg.

45. The use of any one of claims 32 to 43, wherein the compound of Formula (I) or Formula (II) is administered twice daily at a dose selected from the group consisting of 15 mg, 25 mg, 30 mg, 50 mg, 60 mg, 75 mg, 90 mg, 100 mg, 120 mg, 150 mg, 175 mg, 180 mg, 200 mg, 225 mg, 240 mg, 250 mg, 275 mg, 300 mg, 325 mg, 350 mg, 360 mg, 375 mg, and 480 mg.

46. The use of any one of claims 32 to 45, wherein the human subject is treated with the MDM2 inhibitor on days 1-7 of 21-day cycle, wherein on days 8-21 the human is not treated with the MDM2 inhibitor.

47. The use of any one of claims 32 to 46, wherein the compound of Formula (I) or Formula (II) is orally administered.

48. The use of any one of claims 32 to 46, wherein the therapeutically effective amount of the MDM2 inhibitor is 120 mg.

49. The use of any one of claims 32 to 48, the cancer is characterized by overexpression of Bcl-2.

50. The use of any one of claims 32 to 48, the cancer is characterized by overexpression of Bcl-XL.

51. The use of any one of claims 32 to 48, the cancer is characterized by overexpression of Mcl-1.

52. The use of any one of claims 32 to 48, the cancer is characterized by overexpression of Bcl-w.
